(19) United States
(12) Patent Application Publication
(10) Pub. No.: US 2014/0255426 A1
(43) Pub. Date: Sep. 11, 2014

(54) WNT PATHWAY INHIBITORS FOR TREATING VIRAL INFECTIONS

(71) Applicants: Guido Silvestri, Decatur, GA (US); Ann M. Chahroudi, Decatur, GA (US); Mathias Lichterfeld, Boston, MA (US); Maria Jose Buzon, Boston, MA (US); Raymond F. Schinazi, Atlanta, GA (US)

(72) Inventors: Guido Silvestri, Decatur, GA (US); Ann M. Chahroudi, Decatur, GA (US); Mathias Lichterfeld, Boston, MA (US); Maria Jose Buzon, Boston, MA (US); Raymond F. Schinazi, Atlanta, GA (US)

(73) Assignees: EMORY UNIVERSITY, Atlanta, GA (US); Children's Healthcare of Atlanta, Inc., Atlanta, GA (US); The General Hospital Corporation, Boston, MA (US)

(21) Appl. No.: 14/202,459

(22) Filed: Mar. 10, 2014

Related U.S. Application Data
(60) Provisional application No. 61/775,744, filed on Mar. 11, 2013.

(51) Int. Cl. A61K 38/02; A61K 31/427; A61K 45/06; A61K 31/506; A61K 39/42; A61K 31/167; A61K 31/4709; A61K 31/496

(52) U.S. Cl. CPC A61K 38/02; A61K 31/4709; A61K 31/427; A61K 31/496; A61K 31/506; A61K 39/42; A61K 31/167; A61K 45/06
USPC 424/160.1; 514/314; 514/369; 514/253.06; 514/252.18; 514/3.8; 514/622; 514/274; 514/81; 514/230.5; 514/269; 514/365; 514/470; 514/357

(57) ABSTRACT

In certain embodiments, the disclosure relates to methods of treating or preventing a viral infection comprising administering an effective amount of a Wnt pathway inhibitor optionally in combination with one or more anti-viral agents. In certain embodiments, the subject is diagnosed with a chronic viral infection such as human immunodeficiency virus (HIV).

### CROSS REFERENCE TO RELATED APPLICATIONS

This application claims priority to U.S. Provisional Application No. 61/775,744 filed the 11 Mar. 2013, hereby incorporated by reference in its entirety.

### BACKGROUND

Worldwide many people are infected with HIV. Despite the improvement in mortality and morbidity due to antiretroviral therapy (ART), the cost and availability of these drugs places an inordinate burden on individuals and public health systems. For those persons who have access to lifelong ART, reduction of HIV viral loads below detectable limits is often achieved. However, a treatment that can eradicate or functionally cure HIV infection remains elusive due to a persistent reservoir of latently infected cells, and interruption of ART consistently results in a rebound of viremia to pretreatment levels. Thus, there is a need to find improved therapeutic approaches.

Turtle et al. report that a distinct subset of self-renewing human memory CD8⁺ T cells survives cytotoxic chemotherapy. See Immunity, 2009, 31(5):834-44. Chomont et al. report that HIV reservoir persistence is driven by T cell survival and homeostatic proliferation. See Nat Med, 2009, 15(8):893-900. Lugli et al. report memory T cells with stem cell-like properties (T_{SCM} phenotype) that are precursors of central memory, are generated in the acute phase of viral infection, preferentially survive in comparison with other memory cells following elimination of antigen. See J Clin Invest, 2013, 123(2):594-599.

Gattinoni et al. report Wnt signaling arrests effector T cell differentiation and generates CD8⁺ T memory stem cells. See Nat Med, 2009, 15(7):808-13. Jeannet et al. report a role of the Wnt pathway effector T cell factor 1 (Tcf-1) for the establishment of functional CD8⁺ T cell memory. See PNAS, 2010, 107: 9777-9782. Zhou et al. report that differentiation and persistence of memory CD8⁺ T cells depends on T cell factor 1. See Immunity 2010, 33: 229-240.

Kumar et al. report that active β-catenin signaling is an inhibitory pathway for HIV replication. J Virology, 2008, 82(6):2813-2820. See also Modarresi et al., Am J Pathol, 2009, 174(1):123.

References cited herein are not an admission of prior art.

### SUMMARY

In certain embodiments, the disclosure relates to methods of treating or preventing a viral infection comprising administering an effective amount of a Wnt pathway inhibitor optionally in combination with one or more antiviral agents. In certain embodiments, the subject is at risk of, exhibiting symptoms of or diagnosed with a viral infection. In certain embodiments, the subject is diagnosed with a chronic viral infection such as human immunodeficiency virus (HIV) type 1 and 2, as well as other human retroviruses.

In certain embodiments, the Wnt pathway inhibitor is pyrvinium, troglitazone, bosutinib, imatinib, sulindac, niclosamide, XAV-939, non-steroidal anti-inflammatory drug (NSAID), vitamin A, D, COX1, COX2 inhibitor, recombinant peptide, antibody, or a specific binding agent to a Wnt receptor or ligand thereto. A recombinant protein is optionally substituted with polyethylene glycol, saccharide, polysaccharide, carbohydrate or other water solubilizing moiety.

In certain embodiments, the Wnt pathway inhibitor is selected from a secreted frizzled-related protein (SFRP1, SFRP2, SFRP3, SFRP4, SFRP5), Wnt inhibitory factor 1 (Wif1), Cerberus, Sclerostin, Wise, and a Dickkopf family secreted protein (DKK-1, DKK-2, DKK-3, and DKK-4) optionally substituted with polyethylene glycol, saccharide, polysaccharide, carbohydrate or other water solubilizing moiety.

In certain embodiments, the Wnt pathway inhibitor is selected from an antibody to Wnt receptor, ligand, Wnt protein, R-spondin, R-spondin 2 and R-spondin 3 and Norrin.

In certain embodiments, the antiviral agent(s) are selected from abacavir, acyclovir, acyclovir, adefovir, amantadine, amprenavir, ampligen, arbidol, atazanavir, atripla, boceprevir, cidofovir, combivir, complera, darunavir, delavirdine, didanosine, docosanol, dolutegravir, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, stribild, tenofovir, tenofovir disoproxil, tenofovir disoproxil fumarate (TDF), tenofovir alafenamide fumarate (TAF), tipranavir, trifluridine, trizivir, tromantadine, truvada, valaciclovir, valganciclovir, vicriviroc, vidarabine, viramidine, zalcitabine, zanamivir, zidovudine, prodrugs, active metabolites, salts, alternative salts, and combinations thereof.

In certain embodiments, the antiviral agents are emtricitabine, tenofovir DF, and efavirenz. In certain embodiments, the antiviral agents are emtricitabine, tenofovir DF and raltegravir. In certain embodiments, the antiviral agents are emtricitabine, tenofovir DF, ritonavir and darunavir. In certain embodiments, the antiviral agents are emtricitabine, tenofovir DF, ritonavir and atazanavir.

In certain embodiments, the disclosure relates to pharmaceutical compositions comprising a Wnt pathway inhibitor and one or more an antiviral agent.

In certain embodiments, the disclosure relates to kits comprising a Wnt pathway inhibitor and one or more antiviral agents.

### BRIEF DESCRIPTION OF THE FIGURES

FIG. 1A shows data indicating CD4⁺ T_{SCM} cells represent a long-term reservoir for HIV-1 in HAART-treated patients. Proportion of HIV-1 infected cells after ex vivo infection with GFP-encoding R5-tropic or VSV-G pseudotyped HIV-1 (R5: n=17 subjects, VSV-G: n=14 subjects). TNA, naïve T cells; TTD, terminally differentiated T cells. Box and whisker plots indicate the median, interquartile range, and minimum and maximum values.

FIG. 1B shows data indicating cell-associated HIV-1 DNA in sorted CD4⁺ T cell populations (left) and corresponding contributions to the total HIV-1 reservoir in CD4⁺ T cells from HAART-treated individuals (right). Data from n=20 subjects are shown; horizontal lines reflect the median.

FIG. 1C shows left, representative pie charts reflecting the contribution of CD4⁺ T_{SCM} cells to the total viral CD4⁺ T cell reservoir in two persons with large and small HIV-1 reservoirs in total CD4⁺ T cells, respectively. Right, Spearman correlation between contributions of CD4⁺ T_{SCM} cells to the total HIV-1 CD4⁺ T cell reservoir, and corresponding size of the HIV-1 reservoir in total CD4⁺ T cell from all patients shown in 1B.

FIG. 1D shows data indication reactivation of replication-competent HIV-1 from memory CD4⁺ T cell subsets. Orange bars reflect proportions of wells with detectable replication-competent HIV-1; blue bars indicate proportions of wells without detectable replication-competent HIV-1. Numbers above columns reflect total numbers of wells analyzed for each CD4⁺ T cell population; numbers below columns reflect estimated frequencies of cells with replication-competent HIV-1 per million cells (IUPM) based on limiting-dilution analysis. CI, confidence interval.

FIG. 1E shows data from longitudinal evolution studies of HIV-1 DNA in CD4⁺ T cell subsets in n=8 study persons who initiated antiretroviral therapy in primary infection.

FIG. 1F shows pair-wise fold differences in HIV-1 DNA levels measured after short-term and long-term antiretroviral therapy. Mean and s.e.m. from the eight study individuals from 1E are shown.

FIG. 1G shows corresponding contribution of individual CD4⁺ T cell subsets to the total CD4⁺ T cell pool and to the total HIV-1 CD4⁺ T cell HIV-1 reservoir after short-term and long-term antiretroviral therapy.

FIG. 2A shows a phylogenetic analysis of HIV-1 sequences isolated from CD4⁺ T_{SCM} cells. Longitudinal evolution of CD4⁺ T cell counts and viral loads in the three study patients described in the text. Shaded areas reflect periods of antiretroviral treatment exposure. Arrows indicate time of CD4⁺ T cell and plasma sampling. pVL, plasma HIV-1 viral load.

FIG. 2B shows the phylogenetic analysis of HIV-1 sequences longitudinally amplified from sorted CD4⁺ T_{SCM} and T_{CM} cells at the beginning of antiretroviral treatment initiation and after 4-8 years of continuous suppressive therapy in three study persons (respectively FIG. 2A). Identical HIV-1 sequences in T_{SCM} cells (1) and T_{CM} cells (2) are highlighted by gray circles. Circles represent sequences from time point 1; squares represent sequences from time point 2.

FIG. 2C shows circular phylogenetic trees of HIV-1 sequences amplified from the indicated CD4⁺ T cell subsets and from plasma collected at the time points indicated in FIG. 1A (three persons respectively). Gray circles reflect phylogenetic relationships between HIV-1 DNA sequences from CD4⁺ T_{SCM} cells and circulating HIV-1 viral RNA sequences isolated during early untreated disease (3) or during contemporaneous and ensuing time points (4). Identical HIV-1 sequences isolated from CD4⁺ T_{SCM} cells, and from CD4⁺ T_{CM}, T_{EM}, terminally differentiated T cells isolated at later time points, are also highlighted (5).

FIG. 3 shows data on the identification of CD4⁺ T_{SCM} in healthy RM and SM. (A) Flow cytometric analysis of PBMC from a representative SIV-uninfected RM. CD4⁺ T_{SCM} were defined as shown by expression of CD45RA⁺CCR7⁺CD27⁺CD28⁺CD95⁺CD122⁺. (B) Frequencies of circulating CD4⁺ T cell subsets (T_{N}, T_{SCM}, T_{CM}, T_{EM}) in 27 SIV-uninfected RM and 13 SIV-uninfected SM along with the fraction of each subset expressing Ki67 (C), PD-1 (D), and CCR5 (E). T_{N}, T_{CM}, T_{EM} were defined using CD95, CD28 and CCR7: T_{N} (CD28⁺CD95⁻CCR7⁺), T_{CM} (CD45RA⁻CD28⁺CD95⁺CCR7⁺), T_{EM} (CD45RA⁻CD95⁺CCR7⁻), not excluding T_{SCM} from T_{CM} compartment for phenotypic analysis. *p<0.05, **p<0.01, ***p<0.001, ****p<0.0001, NS=not significant (Mann-Whitney). Bars are drawn at the median. (F) Representative CCR5 staining on CD4+TSCM cells from 3 SIV-uninfected RM and SIV-uninfected SM.

FIG. 4 shows data indicating selective preservation of CD4⁺ T_{SCM} cells during pathogenic SIV infection of RM. Frequency (A) and absolute number (B) of total CD3⁺CD4⁺ lymphocytes in PBMC of RM during pathogenic SIV infection. Frequency (C) and absolute number (D) of T_{N}, T_{SCM}, T_{CM}, T_{EM} subsets in PBMC of RM during pathogenic SIV infection. Data in (A) and (C) represent the following RM: 27 SIV-uninfected, 29 acutely infected (day 7-14) 22 early chronic infection (day 65-84), and 19 late chronic infection (day 128-365). Data in (B) and (D) represent the following RM: 16 SIV-uninfected, 18 acutely infected (day 7-14), 11 early chronic (day 65), and 10 late chronic (day 128-365). *p<0.05, **p<0.01, ***p<0.001, ****p<0.0001, NS=not significant (Kruskal-Wallis test, compared to SIV-uninfected). Bars are drawn at the median.

FIG. 5 show data indicating pathogenic SIV infection of RM is associated with significant depletion of CCR5⁺CD4⁺ T_{SCM}. (A) Frequency of total CCR5⁺CD4⁺ T cells as a frequency of CD3⁺ lymphocytes during pathogenic SIV infection of RM. (B) Frequency of CCR5⁺ cells found in each of the four subsets (T_{N}, T_{SCM}, T_{CM}, and T_{EM}). Data in (A) and (B) represent the following RM: 26 SIV-uninfected, 26 acutely infected (day 7-14), 18 early chronic infection (day 65-84), and 19 late chronic infection (day 128-D365). (C) Absolute number of CCR5⁺CD4⁺ T_{SCM} during pathogenic SIV infection of RM per ml of peripheral blood. Data in (C) represent the following RM: 15 SIV-uninfected, 15 acutely infected, 6 early chronic SIV infection, and 10 late chronic SIV infection. *p<0.05, **p<0.01, ***p<0.001, ****p<0.0001, NS=not significant (Kruskal-Wallis, compared to SIV-uninfected). Bars are drawn at the median.

FIG. 6 shows data indicating pathogenic SIV infection of RM is associated with increased proliferation of CD4⁺ T_{SCM} that inversely correlates with the level of CD4⁺ T_{CM}. (A) Frequency of Ki67⁺ T_{N}, T_{SCM}, T_{CM}, T_{EM} in PBMC during pathogenic SIV infection. Data represents the following RM: 22 SIV-uninfected, 29 acutely infected (day 7-14), 22 early chronic infection (day 65-84), and 18 late chronic infection (day 128-365). *p<0.05, **p<0.01, ***p<0.001, ****p<0.0001, NS=not significant (Kruskal-Wallis test, compared to SIV-uninfected). Bars are drawn at the median. (B-D) Correlations of the fraction of Ki67⁺CD4⁺ T_{SCM} and fraction of circulating CD4⁺ T_{SCM}, CD4⁺ T_{CM}, and CD4⁺ T_{EM} during late chronic infection. R and p values were determined by Spearman correlation.

FIG. 7 shows data indicating CD4⁺ T_{SCM} are unperturbed during non-pathogenic SW infection of SM. Comparison of frequency (A) and absolute number (B) of CD4⁺ T_{N}, T_{SCM}, T_{CM}, T_{EM} in SW-uninfected and chronically SIV-infected SM. (C) Frequency of CCR5⁺ T cells in each CD4⁺ T cell subset in SIV-uninfected and chronically SIV-infected SM. (D) Frequency of proliferating CD4⁺ T cell subsets, as measured by Ki67 expression in both uninfected and chronically SIV-infected SM. **p<0.01, NS=not significant (Mann-Whitney). Bars are drawn at the median.

FIG. 8 show data indicating robust levels of CD4⁺ T_{SCM} infection in vivo are observed in SIV-infected RM but not in SIV-infected SM. (A) Fraction of SIV-infected CD4⁺ T_{N}, T_{SCM}, T_{CM}, and T_{EM} cells, as determined by quantitative PCR for the number of SIV gag (RM) or SIV utr (SM) DNA copies per cell in 9 SIVmac251-infected RM, 6 experimentally SIVsmm-infected SM, and 4 naturally SIVsmm-infected SM. Cell number was determined using simultaneous PCR for albumin gene copy number. (B) Plasma viral load of RM and SM shown in (A) as determined by RT-PCR. *p<0.05, **p<0.01, ***p<0.001, NS=not significant (Mann-Whitney test). Bars are drawn at the median.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

Prior to describing the various embodiments, the following definitions are provided and should be used unless otherwise indicated.

“Subject” refers any animal, preferably a human patient, livestock, rodent, monkey or domestic pet.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur atom or replacing an amino group with a hydroxy group. Contemplated derivative include switching carbocyclic, aromatic or phenyl rings with heterocyclic rings or switching heterocyclic rings with carbocyclic, aromatic or phenyl rings, typically of the same ring size. The derivative may be a prodrug such as esters of carboxylic acids or phosphate esters. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze, all hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context may be the same or different and independently hydrogen, halogen hydroxy, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g., patient) is cured and the disease is eradicated. Rather, embodiments of the present disclosure also contemplate treatment that merely reduces symptoms, and/or delays disease progression.

When used in reference to compound(s) disclosed herein, “salts” refer to derivatives of the disclosed compound(s) where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulphur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

As used herein, “heteroaryl” or “heteroaromatic” refers an aromatic heterocarbocycle having 1 to 4 heteroatoms selected from nitrogen, oxygen and sulphur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulphur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkylthio” refers to an alkyl group as defined above attached through a sulphur bridge. An example of an alkylthio is methylthio, (i.e., —S—CH₃).

“Alkoxy” refers to an alkyl group as defined above attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above attached through an amino bridge. An example of an alkylamino is methylamino, (i.e., —NH—CH₃).

“Alkanoyl” refers to an alkyl as defined above attached through a carbonyl bridge (i.e., —(C═O)alkyl).

“Alkylsulfonyl” refers to an alkyl as defined above attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfamoyl” refers to an alkyl as defined above attached through a sulfamoyl bridge (i.e., —S(═O)₂NHalkyl), and an “Arylsulfamoyl” refers to an alkyl attached through a sulfamoyl bridge (i.e., —S(═O)₂NHaryl).

“Alkylsulfinyl” refers to an alkyl as defined above with the indicated number of carbon atoms attached through a sulfinyl bridge (i.e. —S(═O)alkyl).

The terms “halogen” and “halo” refer to fluorine, chlorine, bromine, and iodine.

### Targeting CD4⁺ Memory Stem Cells to Treat HIV Infection

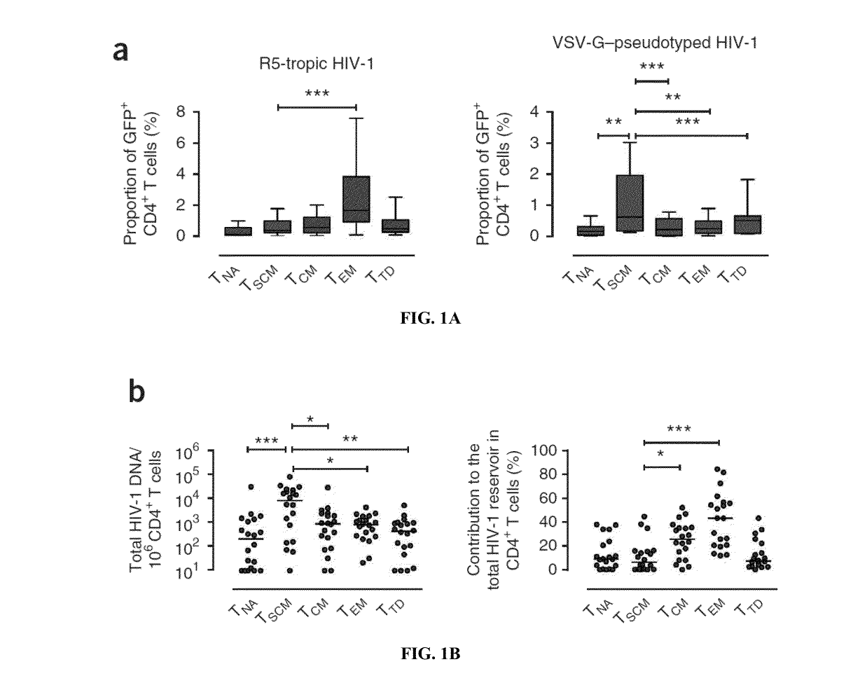

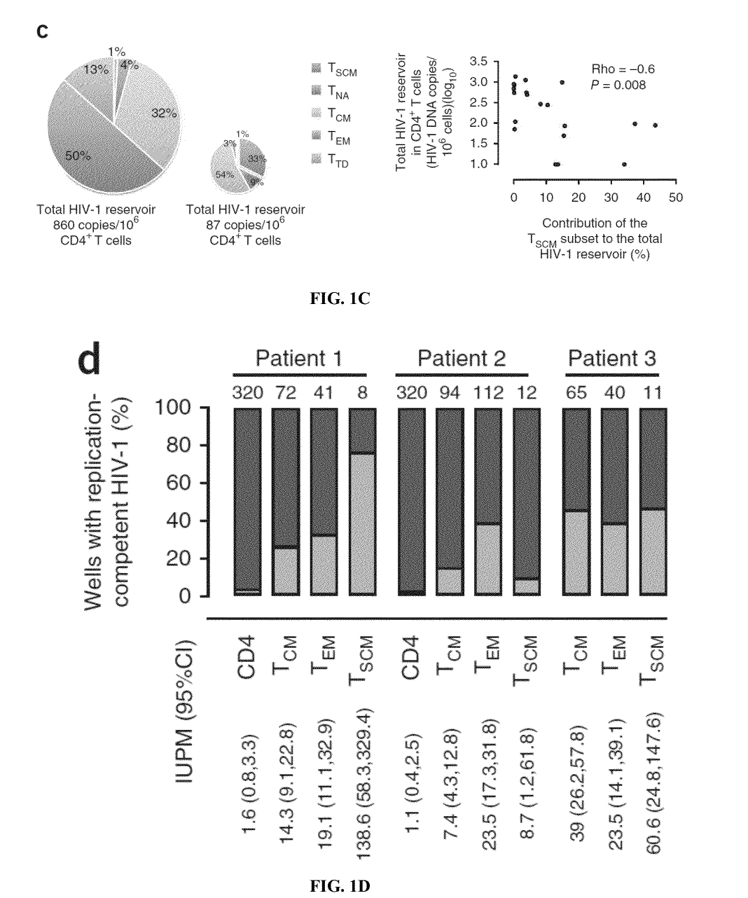

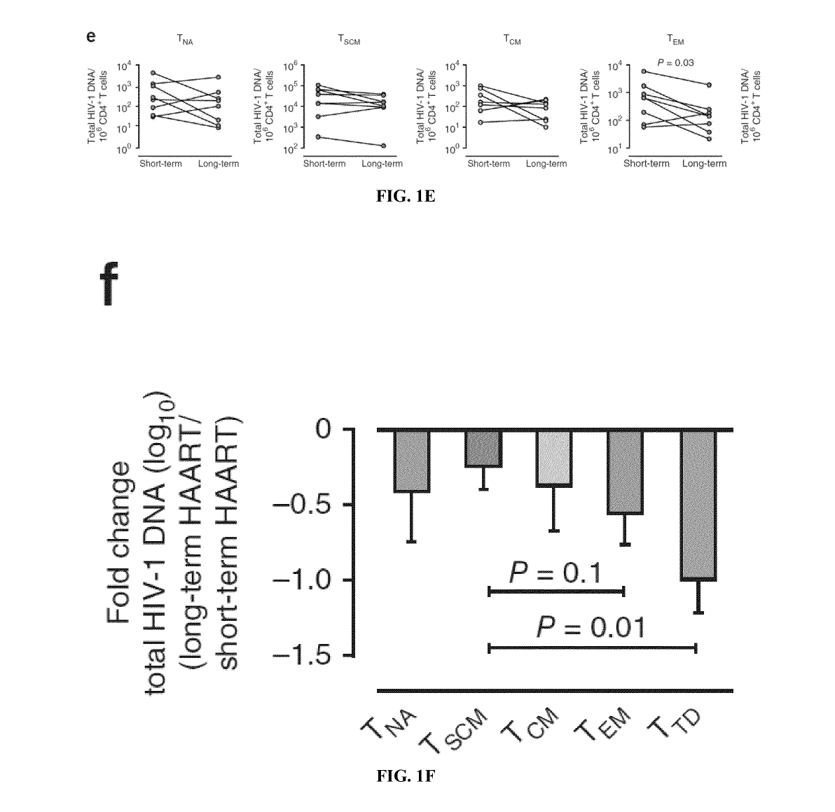

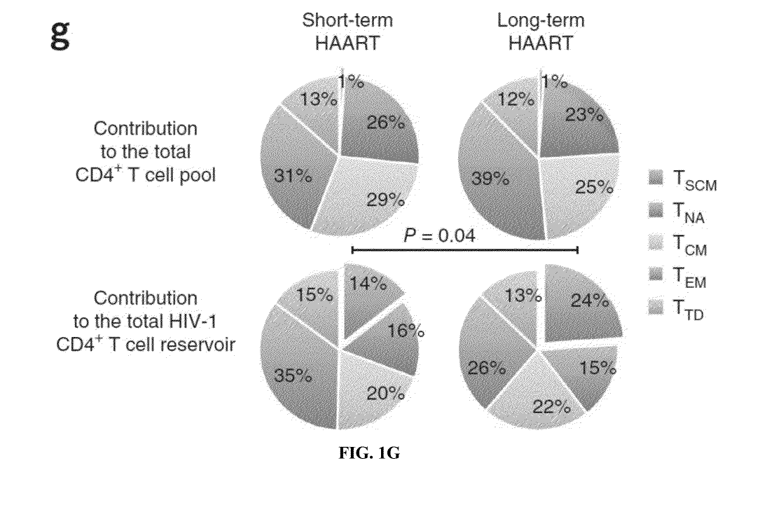

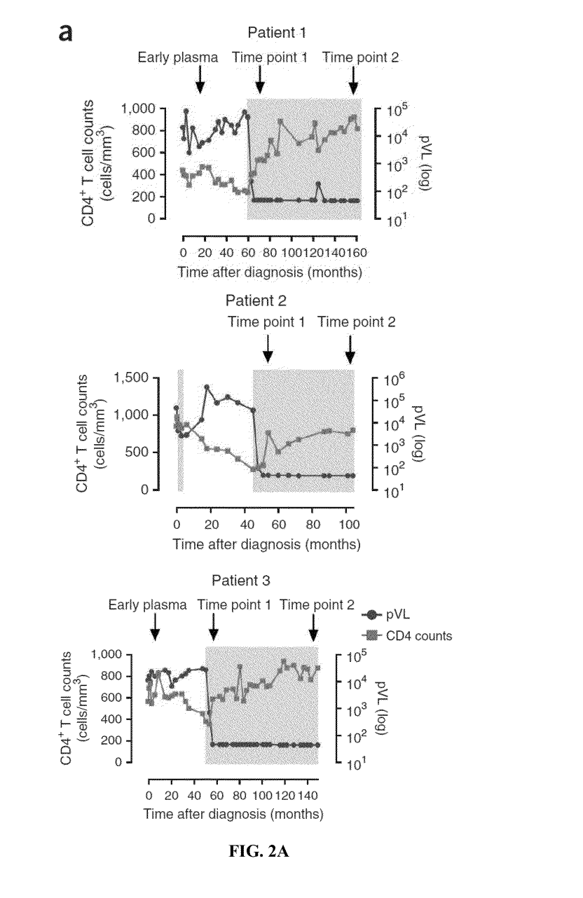

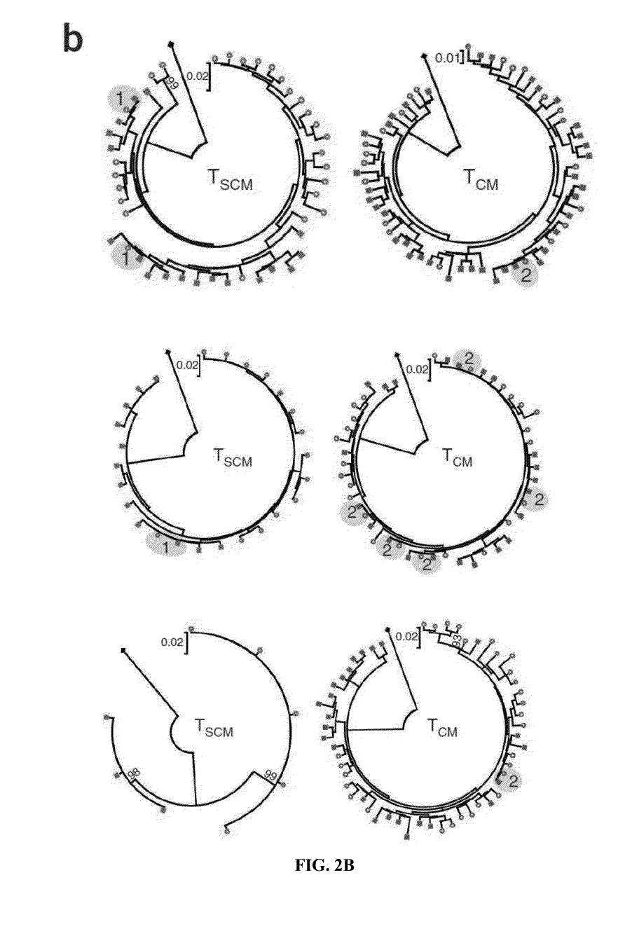

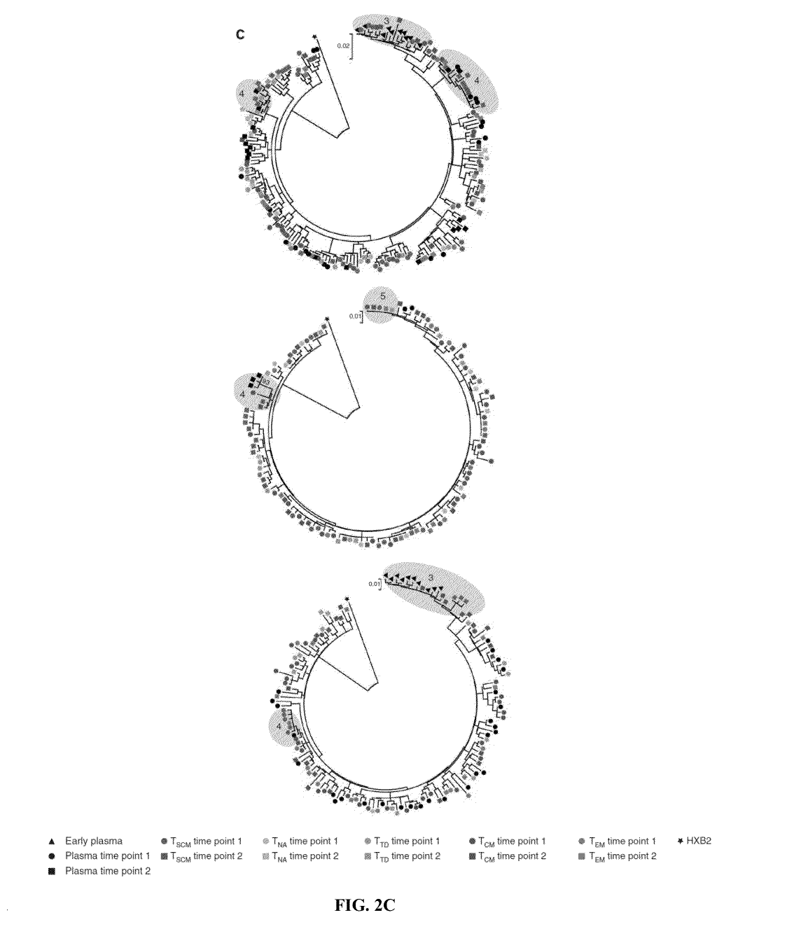

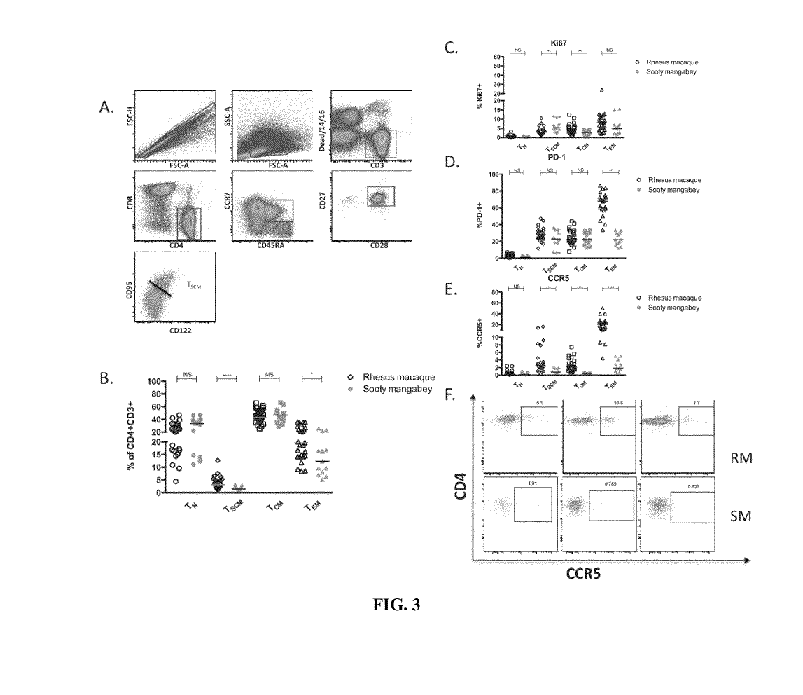

Therapeutic approaches that target the latent viral reservoir have the potential to reduce the need for long-term antiviral treatments, and may ultimately achieve a functional cure for chronic viral infections such as HIV. A reservoir of latently infected cells consists of a small pool of CD4⁺ T cells with a resting memory phenotype. However, data herein indicates that CD4⁺ T memory stem cells (Tscm) are the main cellular contributor to viral persistence over time (FIG. 1). CD4⁺ Tscm are a subset of mature CD4⁺ T cells that are derived from naive T cells following antigen exposure and reversion to a resting state. They possess the stem cell-like properties of enhanced self-renewal and multipotency. Their multipotent capacity is evidenced by one-way differentiation into both central and effector memory T cells following activation. Tscm are phenotypically identified by naïve T cell markers, but are distinguished from nave cells by expression of CD95 and CD122 (IL-2Rβ) (FIG. 2). These cells represent 1-3% of circulating CD4⁺ T cells in humans and rhesus macaques and are also present in lymph nodes. The ability of latently HIV/SIV-infected CD4⁺ Tscm to indefinitely proliferate (and thus transmit integrated virus to daughter cells) makes them an obstacle to the HIV cure effort.

Although it is not intended that certain embodiments be limited by any particular mechanism, it is believed that driving HIV/SIV-infected CD4⁺ Tscm to differentiate into shorter-lived memory subsets will disrupt the continued maintenance of this cellular reservoir by interrupting homeostatic proliferation of CD4⁺ Tscm. By blocking the Wnt signaling pathway that maintains Tscm, in the presence of suppressive antiretroviral therapy, further rounds of infection are inhibited. Activation of the Wnt pathway maintains the “stemness” of Tscm by limiting differentiation. In contrast, inhibition of Wnt signaling is thought to promote programmed differentiation of Tscm into central and effector memory cells. In certain embodiments, this disclosure relates to treating HIV-infected patients with Wnt pathway inhibitors along with suppressive ART as a strategy to eliminate the latent cellular reservoir.

### Wnt Pathway and Inhibitors

Wnt proteins are regulators of cell proliferation and differentiation, and activation of the Wnt pathway is involved in the pathogenesis of several types of human tumors. See Clevers & Nusse, Cell, 2012, 149:1192-1205. Wnt proteins activate several co-receptors on the surface of cells including low-density lipoprotein related protein receptors and Frizzled receptor family members. Wnt binding causes Frizzled to recruit cytosolic dishevelled protein (Dvl1, 2, or 3) leading ultimately to an increased intracellular concentration of the protein β-catenin. β-Catenin is typically in a complex containing axin, adenomatous polyposis coli (APC), glycogen synthase kinase 3 beta (GSK3β), and casein kinase.

Cytosolic β-catenin is degraded upon phosphorylation by a destruction complex containing GSK3β. Inhibition of GSK3β promotes migration of β-catenin to the nucleus increasing activity of T cell factor 1(TCF)/LEF transcription factors. Jeannet et al. report an essential role of the Wnt pathway effector TCF-1 for the establishment of functional CD8+ T cell memory. See PNAS, 2010, 107: 9777-9782. Zhou et al. report that differentiation and persistence of memory CD8⁺ T cells is depend on T cell factor 1. Immunity, 2010, 33: 229-240.

Receptor Tyr kinase-like orphan receptor (ROR), protein Tyr kinase 7 (PTK7), receptor Tyr kinase (RYK), muscle skeletal receptor Tyr kinase (MUSK) are also Wnt receptors and co-receptors. Wnt receptors are regulated by extracellularly by various secreted antagonists, including Cerberus, Dickopf-related protein 1 (DKK1), secreted Frizzled-related protein (SFRP), Sclerostin (and its homologue Wise). R-spondin family and Norrin are Wnt agonists. Wnt inhibitory factor-1 (WIF-1) binds Wnt proteins and acts as a Wnt antagonist. Reducing expression of the WIF-1 activates Wnt signaling.

In certain embodiments, the disclosure contemplates methods of treating or preventing viral infections comprising administering a Wnt pathway inhibitor optionally in combination with one or more antiviral agents. In certain embodiments, the Wnt pathway inhibitor is a small molecule inhibitor such as pyrvinium, troglitazone, bosutinib, imatinib, sulindac, XAV-939, non-steroidal anti-inflammatory drug (NSAID), vitamin A, D, COX1, COX2 inhibitor, recombinant peptide, or antibody to a Wnt receptor or ligand thereto.

In certain embodiments, the Wnt pathway inhibitor is pyrvinium, or salts thereof. Swaraswati et al., report that pyrvinium is a Wnt inhibitor. See PLoS ONE, 2010, 5(11):e1552.1.

In certain embodiments, contemplated Wnt pathway inhibitors are those disclosed in U.S. Patent Application No. 2011/0034441. In certain embodiments, contemplated Wnt pathway inhibitors are 3-(1H-benzo[d]imidazol-2-yl)-1H-indazole, 3-(1H-benzo[d]imidazol-2-yl)-1H-indazole-5-carboxamide, 3-(1H-benzo[d]imidazol-2-yl)-5-(pyridin-3-yl)-1H-indazole, 3-(7-(1H-imidazol-1-yl)-1H-benzo[d]imidazol-2-yl)-5-(pyridin-3-yl)-1H-indazole, N-((4-methyl-5-(3-(7-(4-methyl-1H-imidazol-1-yl)-1H-benzo[d]imidazol-2-yl)-1H-indazol-5-yl)pyridin-2-yl)methyl)ethanamine, 3-(7-(4-methyl-1H-imidazol-1-yl)-1H-benzo[d]imidazol-2-yl)-5-(4-methylpyridin-3-yl)-1H-indazole, or salts, or derivatives thereof, optionally substituted with one or more substituents.

In certain embodiments, the Wnt pathway inhibitor of this disclosure is selected from 1-(4-hydroxy-2-(4-(trifluoromethyl)phenyl)-7,8-dihydropyrido[4,3-d]pyrimidin-6(5H)-yl)ethanone or 2-(4-(trifluoromethyl)phenyl)-7,8-dihydro-5H-thiopyrano[4,3-d]pyrimidin-4-ol (XAV-939), or salts, or derivatives thereof, optionally substituted with one or more substituents. Huang et al. report tankyrase inhibition stabilizes axin and antagonizes Wnt signalling. See Nature 461:614-620, 2009.

In certain embodiments, the Wnt pathway inhibitor of this disclosure is selected from:
4-(1,3-dioxo-3a,4,7,7a-tetrahydro-1H-4,7-methanoisoindol-2(3H)-yl)-N-(quinolin-8-yl)benzamide;
4-(1,3-dioxo-3a,4,7,7a-tetrahydro-1H-4,7-methanoisoindol-2(3H)-yl)-N-(4-methylquinolin-8-yl)benzamide;
4-(1,3-dioxohexahydro-1H-4,7-methanoisoindol-2(3H)-yl)-N-(quinolin-8-yl)cyclohexanecarboxamide;
N-benzyl-4-((1-((6-methyl-4-oxo-4H-pyrido[1,2-a]pyrimidin-2-yl)methyl)-2,4-dioxo-1,2-dihydroquinazolin-3(4H)-yl)methyl)cyclohexanecarboxamide;
N-cyclopentyl-4-((2,4-dioxo-1-((4-oxo-4H-pyrido[1,2-a]pyrimidin-2-yl)methyl)-1,2-dihydroquinazolin-3(4H)-yl)methyl)cyclohexanecarboxamide;
N-benzyl-4-((1-(2-cyanobenzyl)-2,4-dioxo-1,2-dihydroquinazolin-3(4H)-yl)methyl)cyclohexanecarboxamide;
N-(2-((6-methoxybenzo[d]thiazol-2-yl)amino)-2-oxoethyl)-3-(4-methoxyphenyl)-4-oxo-3,4-dihydrophthalazine-1-carboxamide; and
2-((3-(4-fluoro-2-methoxyphenyl)-4-oxo-3,4,6,7-tetrahydrothieno[3,2-d]pyrimidin-2-yl)thio)-N-(6-methoxybenzo[d]thiazol-2-yl)acetamide;

or salts, or derivatives thereof, optionally substituted with one or more substituents. Chen et al. report small molecule-mediated disruption of Wnt-dependent signaling. See Nat Chem Biol, 2009, 5:100-107. See also Willems et al., Circ Res, 2011, 109(4):360-364.

In certain embodiments, the Wnt pathway inhibitor of this disclosure is curcumin or salts, or derivatives thereof, optionally substituted with one or more substituents. Jaiswal et al. report curcumin treatment impairs Wnt signaling. See Oncogene, 2002, 21(55):8414-27.

In certain embodiments, the Wnt pathway inhibitor of this disclosure is 5-chloro-N-(2-chloro-4-nitrophenyl)-2-hydroxybenzamide or salts, or derivatives thereof, optionally substituted with one or more substituents. Chen et al., report the anti-helminthic niclosamide inhibits Wnt/Frizzled1 signaling. See Biochemistry, 2009, 48: 10267-10274.

In certain embodiments, the Wnt pathway inhibitor of this disclosure is resveratrol or salts, or derivatives thereof, optionally substituted with one or more substituents. Hope et al. report resveratrol inhibits Wnt signal. See Mol Nutr Food Res, 2008, 52, Suppl 1: S52 S61.

In certain embodiments, the Wnt pathway inhibitor of this disclosure is selected from:
methyl 1,6,8,14a-tetrahydroxy-11-((4-hydroxy-3,5-dimethoxy-6-methyltetrahydro-2H-pyran-2-yl)methyl)-6a-methoxy-3-methyl-7,9,12,14-tetraoxo-5,6,6a,7,9,12,14,14a-octahydrobenzo[a]tetracene-2-carboxylate;
3-butyryl-1,8-dihydroxy-2-methylphenanthrene-9,10-dione;
1-(4,9-dihydroxy-12-(2-(((4-hydroxyphenoxy)carbonyl)oxy)propyl)-2,6,7,11-tetramethoxy-3,10-dioxo-3,10,12a,12b-tetrahydroperylen-1-yl)propan-2-yl benzoate;
(4,9-dihydroxy-2,6,7,11-tetramethoxy-3,10-dioxo-3,10,12a,12b-tetrahydroperylene-1,12-diyl)bis(propane-2,1-diyl)bis(2,4-dihydroxy-6-methylbenzoate);
5,12-dihydroxy-8,9-bis(2-hydroxypropyl)-7,10-dimethoxyperyleno[1,12-def][1,3]dioxepine-6,11(8aH,8bH)-dione;
2-(6-bromo-1,8-diethyl-1,3,4,9-tetrahydropyrano[3,4-b]indol-1-yl)ethanol;
2-(8-(sec-butyl)-1-ethyl-1,3,4,9-tetrahydropyrano[3,4-b]indol-1-yl)ethanol;
2-(1,8-diethyl-6-phenyl-1,3,4,9-tetrahydropyrano[3,4-b]indol-1-yl)ethanol;
N-benzyl-6-(4-hydroxybenzyl)-8-(naphthalen-1-ylmethyl)-4,7-dioxooctahydro-1H-pyrazino[1,2-a]pyrimidine-1-carboxamide;
N4-(benzo[d][1,3]dioxol-5-ylmethyl)-6-(3-methoxyphenyl)pyrimidine-2,4-diamine;
2-(1-hydroxypentyl)-6-methyl-3-phenethyl-1H-indole-5-carboxylic acid, or salts, or derivatives thereof, optionally substituted with one or more substituents. Kiselyov et al. report small-molecule modulators of Wnt signaling. See Expert Opin Ther Targets, 2007, 11(8):1087-101.

In certain embodiments, the Wnt pathway inhibitor of this disclosure is selected from N-(1-(6-(dimethylamino)nicotinoyl)piperidin-4-yl)-5-(phenylsulfonyl)-2-(trifluoromethyl)benzenesulfonamide and 3-((4-(5-(phenylsulfonyl)-2-(trifluoromethyl)phenylsulfonamido)piperidin-1-yl)sulfonyl)benzoic acid, or salts, or derivatives thereof, optionally substituted with one or more substituents. Moore et al., report modulation of Wnt signaling through inhibition of secreted frizzled-related protein I (sFRP-1) with N-substituted piperidinyl diphenylsulfonyl sulfonamides. See Bioorg Med Chem, 2010, 18(1):190-201.

In certain embodiments, the Wnt pathway inhibitor of this disclosure is 3-(phenylethynyl)-2-(2-(pyridin-2-yl)ethoxy)pyrido[2,3-b]pyrazine, or salts, or derivatives thereof, optionally substituted with one or more substituents. Gong et al. report 3-arylethynyl-substituted pyrido[2,3,-b]pyrazine derivatives as Wnt2/β-catenin pathway inhibitors. See Bioorg Med Chem, 2011, 19(18):5639-5647.

In certain embodiments, the Wnt pathway inhibitor of this disclosure is troglitazone or salts thereof. Wang et al. report thiazolidinediones down-regulate Wnt/beta-catenin signaling. See J Surg Res, 2009, 153(2):210-6.

In certain embodiments, the Wnt pathway inhibitor of this disclosure is a COX1 or COX2 inhibitor or a non-steroidal anti-inflammatory drug (NSAID). In certain embodiments, the Wnt pathway inhibitor of this disclosure is selected from aspirin, ibuprofen, naproxen, sulindac, indomethacin, celecoxib, rofecoxib, 3-nitrooxyphenyl acetylsalicylate, and O²-(acetylsalicyloxymethyl)-1-(pyrrolidin-1-yl)diazen-1-ium-1,2-diolate, or salts thereof. Boon et al. report sulindac targets nuclear beta-catenin accumulation and Wnt signalling. Br. J. Cancer, 2004, 90, 224-229. Other contemplated NSAIDS that are Wnt pathway inhibitors are diflunisal, salsalate, dexibuprofen, fenoprofen, ketoprofen, dexketoprofen, flurbiprofen, oxaprozin, loxoprofen, indomethacin, tolmetin, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid, nimesulide, or salts thereof.

Contemplated COX inhibitors that are Wnt pathway inhibitors include celecoxib, rofecoxib, valdecoxib, parecoxib, lumiracoxib, etoricoxib, licofelone, firocoxib or salts thereof.

In certain embodiments, the Wnt pathway inhibitor of this disclosure is a retinoid, such as retinoic acid, retinol, vitamin D, and or salts, (1R,3S,5Z)-5-[(2E)-2-[(1R,3aS,7aR)-1-[(1R,2E,4E)-6-Ethyl-6-hydroxy-1-methyl-2,4-octadien-1-yl]-octahydro-7α-methyl-4H-inden-4-ylidene]ethylidene]-4-methylene-1,3-cyclohexanediol or derivatives thereof, optionally substituted with one or more substituents. See Barker & Clevers, Nature Reviews Drug Discovery, 2006, 5, 997-1014.

In certain embodiments, the Wnt pathway inhibitor of this disclosure is 5-(((4-(4-methoxyphenyl)-5-(pyridin-4-yl)-4H-1,2,4-triazol-3-yl)thio)methyl)-3-phenyl-1,2,4-oxadiazole, or salts, or derivatives, optionally substituted with one or more substituents. Waaler et al. report synthetic antagonists of Wnt Signaling. See Cancer Res, 2011, 71(1):197-205.

In certain embodiments, the Wnt pathway inhibitor of this disclosure is N-(4-(((4-(4-methoxyphenyl)tetrahydro-2H-pyran-4-yl)methyl)carbamoyl)phenyl)furan-2-carboxamide, or salts, or derivatives, optionally substituted with one or more substituents. Waaler et al. report a tankyrase inhibitor decreases canonical Wnt signaling. See Cancer Res, 2012, 72(11):2822-32.

In certain embodiments, the Wnt pathway inhibitor of this disclosure is 4-(4-(4-fluorophenyl)-5-(2-methoxypyrimidin-4-yl)-1H-imidazol-1-yl)cyclohexanol or 4-(4-(4-fluorophenyl)-2-(4-(methylsulfinyl)phenyl)-1H-imidazol-5-yl)pyridine, or salts, or derivatives thereof, optionally substituted with one or more substituents. Bikkavilli et al. report p38 mitogen-activated protein kinase regulates canonical Wnt-β-catenin signaling by inactivation of GSK3β.

In certain embodiments, the Wnt pathway inhibitor of this disclosure is adenosine 5′-diphosphate (hydroxymethyl) pyrrolidinediol, or salts, or derivatives thereof, optionally substituted with one or more substituents.

In certain embodiments, the Wnt pathway inhibitor of this disclosure is 3-(2,4,6-trimethoxybenzylidene)indolin-2-one or 4-(1-cyclohexyl-4-(4-fluorophenyl)-1H-imidazol-5-yl)pyrimidin-2-amine, or salts, or derivatives thereof, optionally substituted with one or more substituents. Cheong et al., report compounds that induce cell cycle arrest and apoptosis of human cancer cells via CK1delta/epsilon and Wnt/beta-catenin independent inhibition of mitotic spindle formation. See Oncogene, 2011, 30(22): 2558-2569.

In certain embodiments, the Wnt pathway inhibitor of this disclosure is bosutinib or imatinib, or salts thereof. Zhou et al. report tyrosine kinase inhibitor down-regulates the beta-catenin signaling activity. See Cancer Lett 2003; 193(2): 161-170.

In certain embodiments, the Wnt pathway inhibitor of this disclosure is cambinol or filipin or salts thereof.

In certain embodiments, the Wnt pathway inhibitor is selected from a secreted frizzled-related protein (SFRP1, SFRP2, SFRP3, SFRP4, SFRP5), Wnt inhibitory factor 1 (Wif1), Cerberus, Sclerostin, Wise, and a Dickkopf family secreted protein (DKK-1, DKK-2, DKK-3, and DKK-4). In certain embodiments, the Wnt pathway inhibitor of this disclosure is Dishevelled 1 and Dishevelled 2 (Dvl1 and Dvl2). Chen et al. report arrestin1 modulates lymphoid enhancer factor transcriptional activity through interaction with phosphorylated dishevelled proteins. See PNSA, 2001, 98: 14889-14894. See also Chen et al. Science, 2003, 301: 1391-1394.

In certain embodiments, the Wnt pathway inhibitor is selected from an antibody or specific binding agent to a Wnt receptor, Wnt co-receptor, Frizzled, low-density lipoprotein receptor-related protein 5 (LRP5), low-density lipoprotein receptor-related protein 6 (LRP6), receptor Tyr kinase-like orphan receptor 1 (ROR1), receptor Tyr kinase-like orphan receptor 2 (ROR2), protein Tyr kinase 7 (PTK7), receptor Tyr kinase (RYK), muscle skeletal receptor Tyr kinase (MUSK), Syndecans, Syndecan 4, Glypicans, Kremen, LGR4, LGR5, LGR6 and ligands thereto.

In certain embodiments, the Wnt pathway inhibitor is selected from an antibody to Wnt receptor, ligand, Wnt protein, R-spondin, R-spondin 2 and R-spondin 3 and Norrin. In certain embodiments, the Wnt pathway inhibitor of this disclosure is a WNT1 antibody or other specific binding agent. He et al. report a monoclonal antibody against Wnt-1 induces apoptosis in human cancer cells. See Neoplasia 6, 7-14 (2004). In certain embodiments, the Wnt pathway inhibitor of this disclosure is a WNT2 antibody or other specific binding agent. You et al. report an anti-Wnt-2 monoclonal antibody. See Cancer Res. 64, 5385-5389 (2004).

In certain embodiments, the Wnt pathway inhibitor is PRI-724 or salts or derivatives thereof.

In certain embodiments, the Wnt pathway inhibitors is selected from (6S,9S)-N-benzyl-6-(4-hydroxybenzyl)-2,9-dimethyl-4,7-dioxo-8-(quinolin-8-ylmethyl)octahydro-1H-pyrazino[2,1-c][1,2,4]triazine-1-carboxamide, and 4-(((6S,9S)-1-(benzylcarbamoyl)-2,9-dimethyl-4,7-dioxo-8-(quinolin-8-ylme-thyl)octahydro-1H-pyrazino[2,1-c][1,2,4]triazin-6-yl)methyl)phenyl dihydrogen phosphate and (((6S,9S)-N-benzyl-6-(4-hydroxybenzyl)-2,9-dimethyl-4,7-dioxo-8-(quinol-in-8-ylmethyl)octahydro-1H-pyrazino[2,1-c][1,2,4]triazine-1-carboxamide and 4-(((6S,9S)-1-(benzylcarbamoyl)-2,9-dimethyl-4,7-dioxo-8-(quinolin-8-ylmethyl)octahydro-1H-pyrazino[2,1-c][1,2,4]triazin-6-yl)methyl)phenyl dihydrogen phosphate, or salts or derivatives thereof and any compound reported in U.S. Pat. No. 8,455,488, or salts, derivatives thereof.

### Specific Binding Agents and Antibodies

Specific binding agents such as antibodies and antibody fragments that specifically bind Wnt receptor and ligand polypeptides are contemplated as Wnt pathway inhibitors. The antibodies may be polyclonal including mono-specific polyclonal, monoclonal (mAbs), recombinant, chimeric, humanized such as CDR-grafted, human, single chain, catalytic, multi-specific and/or bi-specific, as well as antigen-binding fragments, variants, and/or derivatives thereof.

Once a polynucleotide sequences are identified which encode each chain of the full length monoclonal antibody or the Fab or Fv fragment(s) of the disclosure, host cells, either eukaryotic or prokaryotic, may be used to express the monoclonal antibody polynucleotides using recombinant techniques well known and routinely practiced in the art. Alternatively, transgenic animals are produced wherein a polynucleotide encoding the desired specific binding agent is introduced into the genome of a recipient animal, such as, for example, a mouse, rabbit, goat, or cow, in a manner that permits expression of the polynucleotide molecules encoding a monoclonal antibody or other specific binding agent. In one aspect, the polynucleotides encoding the monoclonal antibody or other specific binding agent can be ligated to mammary-specific regulatory sequences, and the chimeric polynucleotides can be introduced into the germline of the target animal. The resulting transgenic animal then produces the desired antibody in its milk. See Pollock et al., J Immunol, 1999, Meth 231:147-157; Little et al., Immunol Today, 2000, 8:364-370. In addition, plants may be used to express and produce specific binding agents such as monoclonal antibodies by transfecting suitable plants with the polynucleotides encoding the monoclonal antibodies or other specific binding agents.

In another embodiment of the present disclosure, a monoclonal or polyclonal antibody or fragment thereof that is derived from other than a human species may be “humanized” or “chimerized”. Methods for humanizing non-human antibodies are well known in the art. See U.S. Pat. Nos. 5,859,205, 5,585,089, and 5,693,762. Humanization is performed, for example, using methods described in the art by substituting at least a portion of, for example a rodent, complementarity-determining region (CDRs) for the corresponding regions of a human antibody. See Jones et al., Nature, 321: 522-525 (1986); Riechmann et al., Nature, 332: 323-327 (1988); Verhoeyen et al., Science, 239:1534-1536 (1988).

Also encompassed by the disclosure are fully human antibodies as well as, antigen-binding fragments, variants and/or derivatives thereof. Alternatively, transgenic animals (e.g., mice) that are capable of producing a repertoire of human antibodies in the absence of endogenous immunoglobulin production can be used to generate such antibodies. This can be accomplished by immunization of the animal with a Wnt receptor or ligand antigen or fragments thereof where the Wnt receptor or ligand fragments have an amino acid sequence that is unique to Wnt receptor or ligand. Such immunogens can be optionally conjugated to a carrier. See, for example, Jakobovits et al., PNAS, 1993, 90: 2551-2555; Jakobovits et al., Nature, 1993, 362: 255-258; Bruggermann et al., Year in Immuno, 1993, 7: 33. In one method, such transgenic animals are produced by incapacitating the endogenous loci encoding the heavy and light immunoglobulin chains therein, and inserting loci encoding human heavy and light chain proteins into the genome thereof. Partially modified animals, those having less than the full complement of these modifications, are then crossbred to obtain an animal having all of the desired immune system modifications. When administered an immunogen, these transgenic animals are capable of producing antibodies with human variable regions, including human (rather than e.g., murine) amino acid sequences, that are immuno-specific for the desired antigens. See PCT application Nos., PCT/US96/05928 and PCT/US93/06926. Additional methods are described in U.S. Pat. No. 5,545,807, PCT application Nos. PCT/US91/245, PCT/GB89/01207, and in EP 546073B1 and EP 546073A1. Human antibodies may also be produced by the expression of recombinant DNA in host cells or by expression in hybridoma cells as described herein.

Transgenesis is achieved in a number of different ways. See, for example, Bruggeman et al., Immunol Today, 1996, 17:391-7. In one approach, a minilocus is constructed such that gene segments in a germline configuration are brought artificially close to each other. Due to size limitations (i.e., having generally less than 30 kb), the resulting minilocus will contain a limited number of differing gene segments, but is still capable of producing a large repertoire of antibodies. Miniloci containing only human DNA sequences, including promoters and enhancers are fully functional in the transgenic mouse.

When larger numbers of gene segments are desired in the transgenic animal, yeast artificial chromosomes (YACs) are utilized. YACs can range from several hundred kilobases to 1 Mb and are introduced into the mouse (or other appropriate animal) genome via microinjection directly into an egg or via transfer of the YAC into embryonic stem (ES)-cell lines. In general, YACs are transferred into ES cells by lipofection of the purified DNA, or yeast spheroplast fusion wherein the purified DNA is carried in micelles and fusion is carried out in manner similar to hybridoma fusion protocols. Selection of desired ES cells following DNA transfer is accomplished by including on the YAC any of the selectable markers known in the art.

As another alternative, bacteriophage P1 vectors are used which are amplified in a bacterial E. coli host. While these vectors generally carry less inserted DNA than a YAC, the clones are readily grown in high enough yield to permit direct microinjection into a mouse egg. Use of a cocktail of different P1 vectors has been shown to lead to high levels of homologous recombination.

Once an appropriate transgenic mouse (or other appropriate animal) has been identified, using any of the techniques known in the art to detect serum levels of a circulating antibody (e.g., ELISA), the transgenic animal is crossed with a mouse in which the endogenous Ig locus has been disrupted. The result provides progeny wherein essentially all B cells express human antibodies.

As still another alternative, the entire animal Ig locus is replaced with the human Ig locus, wherein the resulting animal expresses only human antibodies. In another approach, portions of the animal's locus are replaced with specific and corresponding regions in the human locus. In certain cases, the animals resulting from this procedure may express chimeric antibodies, as opposed to fully human antibodies, depending on the nature of the replacement in the mouse Ig locus.

Human antibodies can also be produced by exposing human splenocytes (B or T cells) to an antigen in vitro, then reconstituting the exposed cells in an immunocompromised mouse, e.g. SCID or nod/SCID. See Brams et al., J Immunol, 1998, 160: 2051-2058; Carballido et al., Nat Med, 2000, 6: 103-106. In one approach, engraftment of human fetal tissue into SCID mice (SCID-hu) results in long-term hematopoiesis and human T-cell development. See McCune et al., Science, 1988, 241:1532-1639; Ifversen et al., Sem Immunol, 1996, 8:243-248. Any humoral immune response in these chimeric mice is completely dependent on co-development of T-cells in the animals. Martensson et al., Immunol, 1994, 83:1271-179. In an alternative approach, human peripheral blood lymphocytes are transplanted intraperitoneally (or otherwise) into SCID mice. See Mosier et al., Nature, 1988, 335:256-259. When the transplanted cells are treated with either a priming agent, such as Staphylococcal Enterotoxin A (SEA), or anti-human CD40 monoclonal antibodies, higher levels of B cell production are detected.

Alternatively, an entirely synthetic human heavy chain repertoire is created from unrearranged V gene segments by assembling each human V_{H} segment with D segments of random nucleotides together with a human J segment. Likewise, a light chain repertoire is constructed by combining each human V segment with a J segment. Nucleotides encoding the complete antibody (i.e., both heavy and light chains) are linked as a single chain Fv fragment and this polynucleotide is ligated to a nucleotide encoding a filamentous phage minor coat protein. When this fusion protein is expressed on the surface of the phage, a polynucleotide encoding a specific antibody is identified by selection using an immobilized antigen.

In still another approach, antibody fragments are assembled as two Fab fragments by fusion of one chain to a phage protein and secretion of the other into bacterial periplasm. See Hoogenboom et al., Nucl Acids Res, 1991, 19:4133-4137; Barbas et al., PNSA, 1991, 88:7978-7982.

Large-scale production of chimeric, humanized, CDR-grafted, and fully human antibodies, or antigen-binding fragments thereof, are typically produced by recombinant methods. Polynucleotide molecule(s) encoding the heavy and light chains of each antibody or antigen-binding fragments thereof, can be introduced into host cells and expressed using materials and procedures described herein. In a preferred embodiment, the antibodies are produced in mammalian host cells, such as CHO cells.

The specific binding agents of the present disclosure, such as the antibodies, antibody fragments, and antibody derivatives of the disclosure can further comprise any constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In one embodiment, the light or heavy chain constant region is a fragment, derivative, variant, or mutant of a naturally occurring constant region.

In one embodiment, the specific binding agents of the present disclosure, such as the antibodies, antibody fragments, and antibody derivatives of the disclosure comprise an IgG. Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lantto et al., 2002, Methods Mol. Biol. 178:303-16.

The specific binding agents of the present disclosure, such as the antibodies, antibody fragments, and antibody derivatives of the disclosure may comprise the IgG1 heavy chain constant domain or a fragment of the IgG1 heavy chain domain. The antibodies, antibody fragments, and antibody derivatives of the disclosure may further comprise the constant light chain kappa or lambda domains or a fragment of these. Light chain constant regions and polynucleotides encoding them are provided herein below. In another embodiment, the antibodies, antibody fragments, and antibody derivatives of the disclosure further comprise a heavy chain constant domain, or a fragment thereof, such as the IgG2 heavy chain constant region also shown herein below.

The nucleic acid (DNA) encoding constant heavy and constant light chain domains, and the amino acids sequences of heavy and light chain domains are provided herein below. Lambda variable domains can be fused to lambda constant domains and kappa variable domains can be fused to kappa constant domains.

One generates fully human mAbs from nonhuman variable regions using information from the human germline repertoire. Residues within and proximal to CDRs and the V_{H}/V_{L} interface are iteratively explored for substitutions to the closest human germline sequences using semi-automated computational methods. See Bernett et al., J Mol. Biology, 2010, 396(5):1474-1490, hereby incorporated by reference in its entirety. One generates fully human antibodies with substitutions compared to the parent murine sequences. Substitutions may be in the CDRs.

The engineering process to generate fully human mAbs from murine Fvs consists of five main steps: (1) design of framework-optimized V_{H} and V_{L} template sequences (2) identification of the closest matching human germline sequence for the framework-optimized VH and VL, (3) screening of all possible single substitutions that increase the sequence identity of the framework-optimized sequence to the closest human germline sequence, (4) screening of V_{H} and V_{L} variants consisting of combinations of neutral or affinity enhancing single substitutions, and (5) screening of the highest-affinity V_{H} and V_{L} pairs to generate the final fully human mAb.

One defines two principal scores used to measure sequence humanness. Human identity is defined as the number of exact sequence matches between the Fv and the highest identity human germline V_{H}, V_{κ}, J_{H}, and J_{κ} chains (the D-segment for the heavy chain is not included). The second score is the number of total “human 9-mers”, which is an exact count of 9-mer stretches in the Fv that perfectly match any one of the corresponding stretches of nine amino acids in our set of functional human germline sequences. Both human 9-mers and human identity are expressed as percentages throughout in order to enable comparison between antibody Fvs of different lengths.

### Viruses

In some embodiments, the disclosure relates to methods of treating a viral infection comprising administering a Wnt pathway inhibitor to a subject that is diagnosed with, suspected of, or exhibiting symptoms of a viral infection.

Viruses are infectious agents that can typically replicate inside the living cells of organisms. Virus particles (virions) usually consist of nucleic acids, a protein coat, and in some cases an envelope of lipids that surrounds the protein coat. The shapes of viruses range from simple helical and icosahedral forms to more complex structures. Virally coded protein subunits will self-assemble to form a capsid, generally requiring the presence of the virus genome. Complex viruses code for proteins that assist in the construction of their capsid. Proteins associated with nucleic acid are known as nucleoproteins, and the association of viral capsid proteins with viral nucleic acid is called a nucleocapsid.

Viruses are transmitted by a variety of methods including direct or body fluid contact, e.g., blood, tears, semen, preseminal fluid, saliva, milk, vaginal secretions, lesions; droplet contact, fecal-oral contact, or as a result of an animal bite or birth.

A virus has either DNA or RNA genes and is called a DNA virus or a RNA virus respectively. A viral genome is either single-stranded or double-stranded. Some viruses contain a genome that is partially double-stranded and partially single-stranded. For viruses with RNA or single-stranded DNA, the strands are said to be either positive-sense (called the plus-strand) or negative-sense (called the minus-strand), depending on whether it is complementary to the viral messenger RNA (mRNA). Positive-sense viral RNA is identical to viral mRNA and thus can be immediately translated by the host cell. Negative-sense viral RNA is complementary to mRNA and thus must be converted to positive-sense RNA by an RNA polymerase before translation. DNA nomenclature is similar to RNA nomenclature, in that the coding strand for the viral mRNA is complementary to it (negative), and the non-coding strand is a copy of it (positive). Antigenic shift, or reassortment, can result in novel strains. Viruses undergo genetic change by several mechanisms. These include a process called genetic drift where individual bases in the DNA or RNA mutate to other bases. Antigenic shift occurs when there is a major change in the genome of the virus. This can be a result of recombination or reassortment. RNA viruses often exist as quasispecies or swarms of viruses of the same species but with slightly different genome nucleoside sequences.

The genetic material within viruses, and the method by which the material is replicated, vary between different types of viruses. The genome replication of most DNA viruses takes place in the nucleus of a cell. If the cell has the appropriate receptor on its surface, these viruses enter the cell by fusion with the cell membrane or by endocytosis. Most DNA viruses are entirely dependent on the host DNA and RNA synthesizing machinery, and RNA processing machinery. Replication usually takes place in the cytoplasm. RNA viruses typically use their own RNA replicase enzymes to create copies of their genomes.

The Baltimore classification of viruses is based on the mechanism of mRNA production. Viruses must generate mRNAs from their genomes to produce proteins and replicate themselves, but different mechanisms are used to achieve this. Viral genomes may be single-stranded (ss) or double-stranded (ds), RNA or DNA, and may or may not use reverse transcriptase (RT). Additionally, ssRNA viruses may be either sense (plus) or antisense (minus). This classification places viruses into seven groups: I, dsDNA viruses (e.g. adenoviruses, herpesviruses, poxviruses); II, ssDNA viruses (plus)sense DNA (e.g. parvoviruses); III, dsRNA viruses (e.g. reoviruses); IV, (plus)ssRNA viruses (plus)sense RNA (e.g. picornaviruses, togaviruses); V, (minus)ssRNA viruses (minus)sense RNA (e.g. orthomyxoviruses, Rhabdoviruses); VI, ssRNA-RT viruses (plus)sense RNA with DNA intermediate in life-cycle (e.g. retroviruses); and VII, dsDNA-RT viruses (e.g. hepadnaviruses).

In certain embodiments, the subject is diagnosed to have a virus by nucleic acid detection or viral antigen detection.

In certain embodiments, the subject is diagnosed with influenza A virus including subtype H1N1, influenza B virus, influenza C virus, rotavirus A, rotavirus B, rotavirus C, rotavirus D, rotavirus E, SARS coronavirus, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), yellow fever virus, measles virus, mumps virus, respiratory syncytial virus, rinderpest virus, California encephalitis virus, hantavirus, rabies virus, ebola virus, marburg virus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis C, hepatitis D, or hepatitis E, HIV-1, and HIV-2.

### Human Immunodeficiency Virus (HIV)

HIV is a lentivirus (a member of the retrovirus family) that causes acquired immunodeficiency syndrome (AIDS). Lentiviruses are transmitted as single-stranded, positive-sense, enveloped RNA viruses. Upon entry of the target cell, the viral RNA genome is converted to double-stranded DNA by a virally encoded reverse transcriptase. This viral DNA is then integrated into the cellular DNA by a virally encoded integrase, along with host cellular co-factors. There are two species of HIV. HIV-1 is sometimes termed LAV or HTLV-III.

HIV infects primarily vital cells in the human immune system such as helper T cells (CD4⁺ T cells), macrophages, and dendritic cells. HIV infection leads to low levels of CD4⁺ T cells. When CD4⁺ T cell numbers decline below a critical level, cell-mediated immunity is lost, and the body becomes progressively more susceptible to other viral or bacterial infections. Subjects with HIV typically develop malignancies associated with the progressive failure of the immune system.

The viral envelope is composed of two layers of phospholipids taken from the membrane of a human cell when a newly formed virus particle buds from the cell. Embedded in the viral envelope are proteins from the host cell and a HIV protein known as Env. Env contains glycoproteins gp120, and gp41. The RNA genome consists of structural landmarks (LTR, TAR, RRE, PE, SLIP, CRS, and INS) and nine genes (gag, pol, and env, tat, rev, nef, vif, vpr, vpu, and sometimes a tenth tev, which is a fusion of tat env and rev) encoding 19 proteins. Three of these genes, gag, pol, and env, contain information needed to make the structural proteins for new virus particles. HIV-1 diagnosis is typically done with antibodies in an ELISA, Western blot, or immunoaffinity assays or by nucleic acid testing (e.g., viral RNA or DNA amplification).

HIV is typically treated with a combination of antiviral agent, e.g., two nucleoside-analogue reverse transcription inhibitors and one non-nucleoside-analogue reverse transcription inhibitor or protease inhibitor. The three drug combination is commonly known as a triple cocktail. In certain embodiments, the disclosure relates to treating a subject diagnosed with HIV by administering Wnt signaling pathway inhibitor in combination with two nucleoside-analogue reverse transcription inhibitors and one non-nucleoside-analogue reverse transcription inhibitor or protease inhibitor.

In certain embodiments, the disclosure relates to treating a subject by administering Wnt signaling pathway inhibitor, emtricitabine, tenofovir DF, and efavirenz. In certain embodiments, the disclosure relates to treating a subject by administering Wnt signaling pathway inhibitor, emtricitabine, tenofovir DF and raltegravir. In certain embodiments, the disclosure relates to treating a subject by administering Wnt signaling pathway inhibitor, emtricitabine, tenofovir DF or TAF, ritonavir and darunavir. In certain embodiments, the disclosure relates to treating a subject by administering Wnt signaling pathway inhibitor, emtricitabine, tenofovir DF or TAF, ritonavir and atazanavir.

Banana lectin (BanLec or BanLec-1) is one of the predominant proteins in the pulp of ripe bananas and has binding specificity for mannose and mannose-containing oligosaccharides. BanLec binds to the HIV-1 envelope protein gp120. In certain embodiments, the disclosure relates to treating viral infections, such as HIV, by administering a Wnt signaling pathway inhibitor in combination with a banana lectin.

### Combination Therapies

In some embodiments, the disclosure relates to treating a viral infection by administering a Wnt signaling pathway inhibitor in combination with a second antiviral agent. In further embodiments, Wnt signaling pathway inhibitor is administered in combination with one or more of the following agents: abacavir, acyclovir, acyclovir, adefovir, amantadine, amprenavir, ampligen, arbidol, atazanavir, atripla, boceprevir, cidofovir, combivir, complera, darunavir, delavirdine, didanosine, docosanol, dolutegravir, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, stribild, tenofovir, tenofovir disoproxil, tenofovir alafenamide fumarate (TAF), tipranavir, trifluridine, trizivir, tromantadine, truvada, valaciclovir, valganciclovir, vicriviroc, vidarabine, viramidine, zalcitabine, zanamivir, or zidovudine, and combinations thereof.

Antiviral agents include, but are not limited to, protease inhibitors (PIs), integrase inhibitors, entry inhibitors (fusion inhibitors), maturation inhibitors, and reverse transcriptase inhibitors (antiretrovirals). Combinations of antiviral agents create multiple obstacles to viral replication, i.e., to keep the number of offspring low and reduce the possibility of a superior mutation. If a mutation that conveys resistance to one of the agents being taken arises, the other agents continue to suppress reproduction of that mutation. For example, a single anti-retroviral agent has not been demonstrated to suppress an HIV infection for long. These agents are typically taken in combinations in order to have a lasting effect. As a result, the standard of care is to use combinations of antiretrovirals.

Reverse transcribing viruses replicate using reverse transcription, i.e., the formation of DNA from an RNA template. Retroviruses often integrate the DNA produced by reverse transcription into the host genome. They are susceptible to antiviral drugs that inhibit the reverse transcriptase enzyme. In certain embodiments the disclosure relates to methods of treating viral infections by administering a Wnt signaling pathway inhibitor and a retroviral agent such as nucleoside and nucleotide reverse transcriptase inhibitors (NRTI) and/or a non-nucleoside reverse transcriptase inhibitors (NNRTI). Examples of nucleoside reverse transcriptase inhibitors include zidovudine, didanosine, zalcitabine, stavudine, lamivudine, abacavir, emtricitabine, entecavir, apricitabine. Examples of nucleotide reverse transcriptase inhibitors include tenofovir and adefovir in their phosphorylated, diphosphorylated, triphosphorylated forms and derivatives. Examples of non-nucleoside reverse transcriptase inhibitors include efavirenz, nevirapine, delavirdine, and etravirine.

In certain embodiments, the disclosure relates to methods of treating a viral infection by administering a Wnt signaling pathway inhibitor in combination with an antiviral drug, e.g., 2′,3′-dideoxyinosine and a cytostatic agent, e.g., hydroxyurea.

Human immunoglobulin G (IgG) antibodies are believed to have opsonizing and neutralizing effects against certain viruses. IgG is sometimes administered to a subject diagnosed with immune thrombocytopenic purpura (ITP) secondary to a viral infection since certain viruses such as, HIV and hepatitis, cause ITP. In certain embodiments, the disclosure relates to methods of treating or preventing viral infections comprising administering a Wnt signaling pathway inhibitor in combination with an immunoglobulin to a subject. IgG is typically manufactured from large pools of human plasma that are screened to reduce the risk of undesired virus transmission. The Fc and Fab functions of the IgG molecule are usually retained. Therapeutic IgGs include Privigen, Hizentra, and WinRho. WinRho is an immunoglobulin (IgG) fraction containing antibodies to the Rho(D) antigen (D antigen). The antibodies have been shown to increase platelet counts in Rho(D) positive subjects with ITP. The mechanism is thought to be due to the formation of anti-Rho(D) (anti-D)-coated RBC complexes resulting in Fc receptor blockade, thus sparing antibody-coated platelets

### EXAMPLES

### HIV-1 Persistence in CD4+ T Cells with Stem Cell-Like Properties

Antiretroviral combination therapy effectively suppresses HIV-1 replication, but replication-competent virus can persist in memory CD4⁺ T cells despite treatment. The memory CD4⁺ T cell compartment includes long-lasting central memory (T_{CM}) cells, which undergo a sequential developmental program with progressive commitment to more differentiated, short-lived effector memory (TEM) and terminally differentiated T cell types. The presence of a more immature memory T cell population with stem cell-like properties has previously been hypothesized on the basis of animal studies, and recently, small proportions of T cells with stem cell characteristics have been discovered in humans, mice and nonhuman primate. These cells, termed T_{SCM} cells, seem to represent the earliest and most long-lasting developmental stage of memory T cells, and can differentiate into large numbers of T_{CM}, T_{EM} and terminally differentiated T cells while maintaining their own pool size through homeostatic self-renewal. It is thought that HIV-1 can use CD4⁺ T_{SCM} cells as a preferred niche for promoting long-term viral persistence.

To test this concept, the susceptibility of CD4⁺ T_{SCM} cells to HIV-1 infection was investigated. These experiments demonstrated that CD4⁺ T_{SCM} cells, phenotypically defined as described in previous studies, were approximately as susceptible as CD4⁺ T_{CM} cells to infection with an R5-tropic HIV-1 isolate (FIG. 1a), although their surface expression of CCR5 was slightly lower. In addition, CD4⁺ T_{SCM} cells were highly susceptible to infection with a vesicular stomatitis virus G protein (VSV-G)-pseudotyped HIV-1 viral particles (FIG. 1a), despite their comparatively low expression of T cell activation makers. HIV-1 RNA was readily detectable in CD4⁺ T_{SCM} cells from untreated HIV-1-infected patients. CD4⁺ T_{SCM} cells had low sensitivity to the cytopathic effects associated with HIV-1 infection and expressed reduced levels of the cell-intrinsic HIV-1 restriction factors TRIM5α, APOBEC3G and SAMHD1. Together, these data indicate that CD4⁺ T_{SCM} cells are permissive to HIV-1 infection and can serve as physiologic target cells for HIV-1.

The levels of HIV-1 DNA were determined in sorted CD4⁺ T_{SCM} cells from HIV-1 infected patients who had been treated with suppressive highly active antiretroviral therapy (HAART) for a median of 7 years. The proportions of CD4⁺ T cells in these patients did not differ from those in an HIV-1-negative control cohort. In these HAART-treated patients, per-cell levels of HIV-1 DNA were highest in CD4⁺ T_{SCM} cells, but their average contribution to the total viral reservoir in CD4⁺ T cells was only approximately 8% (FIG. 1b). Notably, in this cross-sectional analysis, the contribution of CD4⁺ T_{SCM} cells to the total viral reservoir in CD4⁺ T cells varied considerably among different HAART-treated patients and was inversely associated with HIV-1 DNA levels in the entire CD4⁺ T cell compartment (FIG. 1c). This negative association was observed selectively in the CD4⁺ T_{SCM} cell compartment, and it resulted in a disproportionately increased contribution of CD4⁺ T_{SCM} cells to the total viral CD4 T⁺ cell reservoir in patients with a smaller viral reservoir in CD4⁺ T_{CM} and T_{EM} cells. This suggests that HIV-1-infected CD4⁺ T_{SCM} cells represent a not necessarily large but very stable and durable component of the viral CD4⁺ T cell reservoir that becomes increasingly prominent when viral reservoirs in alternative CD4⁺ T cell subsets are limited. HIV-1 DNA was also detectable in CD4⁺ T_{SCM} cells from elite controllers, a small group of HIV-1-infected individuals who maintain undetectable levels of HIV-1 replication in the absence of antiretroviral therapy, although at significantly lower levels than in CD4⁺ T_{SCM} cells from HAART-treated patients.

As only a small proportion of CD4⁺ T cell-associated HIV-1 DNA encodes replication-competent virus, viral outgrowth assays were performed from three study subjects who had been on continuous suppressive antiretroviral combination therapy with HAART for a median of 28 months (range 14-42 months). Replication-competent virus were retrieved from CD4⁺ T_{SCM} cells in all three cases, and the estimated frequency of cells harboring replication-competent HIV-1 in CD4⁺ T_{SCM} cells exceeded the corresponding frequencies in CD4⁺ T_{CM} and TEM cells in two of the three patients (FIG. 1d). These findings indicate that HIV-1 DNA in CD4⁺ T_{SCM} cells is functionally capable of resuming active viral gene expression.

Given their stem cell-like properties, CD4⁺ T_{SCM} cells may represent a privileged site for long-term viral persistence. To better investigate this, HIV-1 DNA in sorted CD4+ T cell subsets were longitudinally analyzed from eight individuals who started antiretroviral therapy during primary infection and then remained on suppressive antiretroviral therapy without treatment interruptions. Using pair-wise comparisons between cell-associated HIV-1 DNA during earlier stages of antiretroviral therapy (median of 1 year, range: 10-14 months) and during later stages of treatment (median of 9 years, range 7-11 years), stable or mildly decreasing viral DNA

levels in CD4⁺ T_{SCM} cells were observed; the viral DNA decline in CD4⁺ T_{CM} and naive CD4+ T cells was slightly more pronounced (FIG. 1e). In contrast, in the more short-lived CD4⁺ T_{EM} and terminally differentiated T cell populations, a significant reduction in per-cell levels of total HIV-1 DNA was noticed over time (FIG. 1e). Notably, among all CD4⁺ T cell subsets, the relative longitudinal decline in total HIV-1 DNA at per-cell levels was smallest in CD4⁺ T_{SCM} cells, although differences between CD4⁺ T_{SCM} cells and naive CD4⁺ T cells and T_{CM} cells did not reach statistical significance in our small study cohort (FIG. 1f). Of note, CD4⁺ T_{SCM} cells made a relatively small contribution to the total CD4⁺ T cell reservoir after the first year of suppressive antiretroviral therapy (FIG. 1g). Yet, after long-term antiretroviral treatment, there was a significant increase in the contribution of CD4⁺ T_{SCM} cells to the total viral reservoir in CD4⁺ T cells, despite the fact that the numeric contribution of CD4⁺ T_{SCM} cells to the total CD4⁺ T cell pool did not change. The contribution of CD4⁺ T_{CM} cells to the total viral CD4⁺ T cell reservoir also slightly increased over time, but this did not reach the level of statistical significance. In contrast, the contribution of CD4⁺ T_{EM} cells to the viral CD4⁺ T cell reservoir declined, despite a numerically increased proportion of T_{EM} cells in the total CD4⁺ T cell pool (FIG. 1g). These data indicates that CD4⁺ T_{SCM} cells can support long-term viral persistence in patients treated with HAART.

The proviral Env gene in DNA samples isolated from longitudinally sorted CD4⁺ T cell subsets was subsequently sequenced from three HIV-1-infected patients who did not receive antiretroviral therapy during the initial years of disease, followed by continuous treatment with suppressive antiretroviral agents (FIG. 2a). Substantial intraindividual variability was observed between viral sequences from CD4⁺ T_{SCM} cells collected at the beginning of antiretroviral therapy and several years later, probably reflecting sampling of cells infected with different circulating viral strains during early disease stages (FIG. 2b).

Yet, in CD4⁺ T_{SCM} and CD4⁺ T_{CM} cells (which were sampled in approximately 10- to 30-fold higher frequencies than CD4⁺ T_{SCM} cells), several identical HIV-1 sequences in samples collected at the beginning of antiretroviral therapy were noticed and after 4-8 years of continuous treatment, which is consistent with long-term viral persistence in these CD4⁺ T cell subsets (FIG. 2b). Notably, identical proviral sequences during early and later stages of antiretroviral therapy were not detected in naive or more terminally differentiated CD4⁺ T cell subsets. Phylogenetic relationships between proviral HIV-1 Env sequences from CD4⁺ T_{SCM} cells were analyzed and circulating viral RNA amplified from plasma samples collected during early untreated disease stages and from residual HIV-1 viremia at the time of suppressive antiretroviral therapy. Among all viral sequences from CD4⁺ T cell subsets collected during suppressive antiretroviral therapy at later disease stages (6-12 years after infection), HIV-1 DNA isolated from CD4⁺ T_{SCM} and T_{CM} cells was phylogenetically most closely related to circulating plasma sequences from early infection; this suggests that the viral strains circulating in early disease seem more likely to persist long term upon infection of CD4⁺ T_{CM} and T_{SCM} cell subsets (FIG. 2c). In addition, pair-wise sequence comparisons revealed that the genetic distance between early plasma HIV-1 RNA sequences and HIV-1 DNA sequences from CD4⁺ T cell subsets collected during later stages of infection was lowest for HIV-1 DNA sequences from CD4⁺ T_{SCM} and CD4⁺ T_{CM} cells. Sequences from CD4⁺ T_{SCM} cells also showed phylogenetic associations with contemporaneous and ensuing sequences isolated from plasma during suppressive antiretroviral therapy, which is consistent with a possible interchange between viral strains in CD4₊ T_{SCM} cells and circulating viral species (FIG. 2c). Finally, viral sequences from CD4⁺ T_{SCM} cells at early stages of antiretroviral therapy were noted that were identical to those from CD4⁺ T_{CM}, T_{EM} and terminally differentiated T cells isolated several years later, supporting the role of CD4⁺T_{SCM} cells as precursor cells for more differentiated CD4₊ T cell subsets (FIG. 2c). Although these phylogenetic studies were performed in a limited number of patients, they suggest that CD4⁺ T_{SCM} and T_{CM} cells may comprise a long-term reservoir for HIV-1.

These studies indicate that CD4⁺ T_{SCM} cells, despite their low frequencies, stand out among other memory CD4⁺ T cell subsets as the cell population in which long-term HIV-1 persistence is particularly evident, probably owing to intrinsic cellular programs of these cells that give them superior abilities to self-renew, resist apoptosis and survive for extremely long periods of time.

Pharmaceutical inhibition of stem cell-specific molecular pathways is being investigated for targeting cancer stem cells, and the specific targeting of cellular pathways responsible for the stem cell-like properties of CD4⁺ T_{SCM} cells may also have adjunct or additive effects on reducing the persistence of HIV-1-infected CD4⁺ T_{SCM} cells. Thus, our increasing understanding of how stem cell-like properties of cellular immune memory maintain HIV-1 persistence despite HAART may be translatable into improved clinical strategies for inducing HIV-1 eradication and cure.

### Identification of CD4⁺ T_{SCM} in Healthy Rhesus Macaques and Sooty Mangabeys

Pathogenic HIV infection of humans and SIV infection of rhesus macaques (RM) are characterized by progressive depletion of CD4⁺ T cells and development of a lethal state of immunodeficiency termed AIDS. In contrast, SIV infection of African nonhuman primate species that are natural hosts for the virus, such as the sooty mangabeys (SM) and the African green monkeys (AGM), are typically nonpathogenic despite high virus replication.

CD4⁺ and CD8⁺ TSCM have been phenotypically identified in humans as CD45RA⁺CD45RO⁻CD62L⁺CCR7⁺CD27⁺CD28⁺CD127⁺CD95⁺CD122⁺, and in RM and pigtailed macaques are defined as CD45RA⁺CCR7⁺CD27⁺CD28⁺CD127⁺CD95⁺. This immunophenotypic definition was confirmed in healthy RM (see FIG. 3A for gating strategy) and SM. CD4⁺ T_{SCM} isolated from both RM and SM expressed intermediate levels of CXCR3 and LFA-1 that were between those of naive and central memory CD4⁺ T cells. In healthy RM, it was confirmed that CD4⁺ T_{SCM} can be readily identified in the blood, lymph nodes, bone marrow, and spleen, but are present at a lower frequency in the intestinal mucosa. The levels of circulating CD4⁺ T_{SCM} in healthy SIV-uninfected RM and SM were compared as percentage of total CD4⁺ T cells. As shown in FIG. 3B, the percentage of CD4⁺ T_{SCM} ranged between 1-8% in RM and 0.5-3% in SM, with the levels observed in RM being significantly higher (p=0.0004). Interestingly, CD4⁺ T_{SCM} from SIV-uninfected SM show higher levels of proliferation (measured as expression of Ki67) as compared to RM (p=0.0034, FIG. 3C), perhaps suggesting that a relatively smaller pool of CD4⁺ T_{SCM} maintains CD4⁺ memory T cell homeostasis through higher baseline rates of proliferation in SM. We also found that CD4⁺ T_{SCM} from RM also express slightly higher levels of the inhibitory marker PD-1 as compared to CD4⁺ T_{SCM} from SM, though this trend was not statistically significant (FIG. 3D).

CD4⁺ T cells of both SIV-infected and uninfected SM express lower levels of the SW coreceptor CCR5 than CD4⁺ T cells of humans and RM. This difference is particularly evident for CD4⁺ T_{CM}. The levels of CCR5 expression on CD4⁺ T_{SCM} from healthy RM and SM wew next examined. Significantly higher percentages of CCR5⁺CD4⁺ T_{SCM} from RM compared to SM were found (p=0.0009, FIG. 3E). Three representative examples of CCR5 staining on CD4⁺ T_{SCM} are shown in FIG. 3F, which emphasize the almost complete absence of CCR5 on CD4⁺ T_{SCM} of SM.

### Numeric Preservation of CD4+ TSCM During Pathogenic SIV Infection of RM

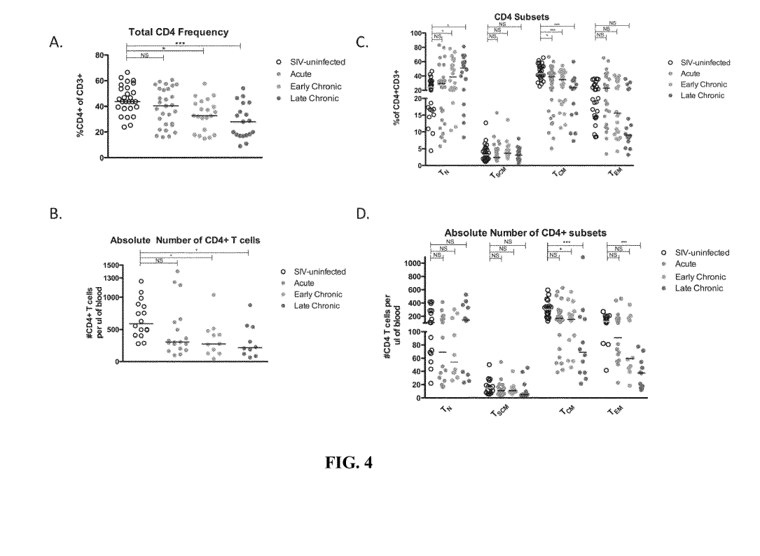

Pathogenic SIV infection of RM is characterized by a progressive depletion of CD4⁺ T cells from blood and mucosal tissues, which is typically associated with the loss of CD4⁺ T_{CM} homeostasis. To examine the dynamics of CD4⁺ T_{SCM} during pathogenic SW infection of RM, a total of 51 RM were examined, including healthy SIV-uninfected animals and SIV-infected animals at different stages of infection. The animals studied here exhibited the well-characterized progressive depletion of circulating CD4⁺ T cells associated with SIV infection, measured as the fraction of CD3⁺ T lymphocytes (FIG. 4A) or absolute number of cells per micro liter of blood (FIG. 4B). In the same animals, the relative distribution of four key CD4+ T cell subsets were examined, i.e., naive (CD28⁺CD95⁻); CD4⁺ T_{SCM} (CD45RA⁺CCR7⁺CD27⁺CD28⁺CD95⁺CD122⁺); CD4⁺ T_{CM} (CD28⁺CD95⁺CCR7⁺); and CD4⁺ T_{EM} (CD95⁺CCR7⁻). Levels of both CD4⁺ T_{CM} and T_{EM} (primary targets for SIV infection) were altered by SIV infection, with a significant decline of the fraction and absolute number of CD4⁺ T_{CM} in both early and late chronic SIV infection (p<0.01 and p<0.001, respectively, FIGS. 4C-D), and a significant decline of the absolute number of CD4⁺ T_{EM} in late chronic SIV infection (p<0.001, FIG. 4D). Interestingly, neither the fraction nor the absolute number of CD4⁺ T_{SCM} was decreased in either acute or chronic SIV infection of RM (FIG. 4C-D). As such, these data indicate that pathogenic SIV infection of RM is not associated with a significant numerical decline of circulating CD4⁺ T_{SCM}.

Pathogenic SIV Infection of RM is Associated with a Significant Depletion of CCR5⁺CD4⁺ T_{SCM}

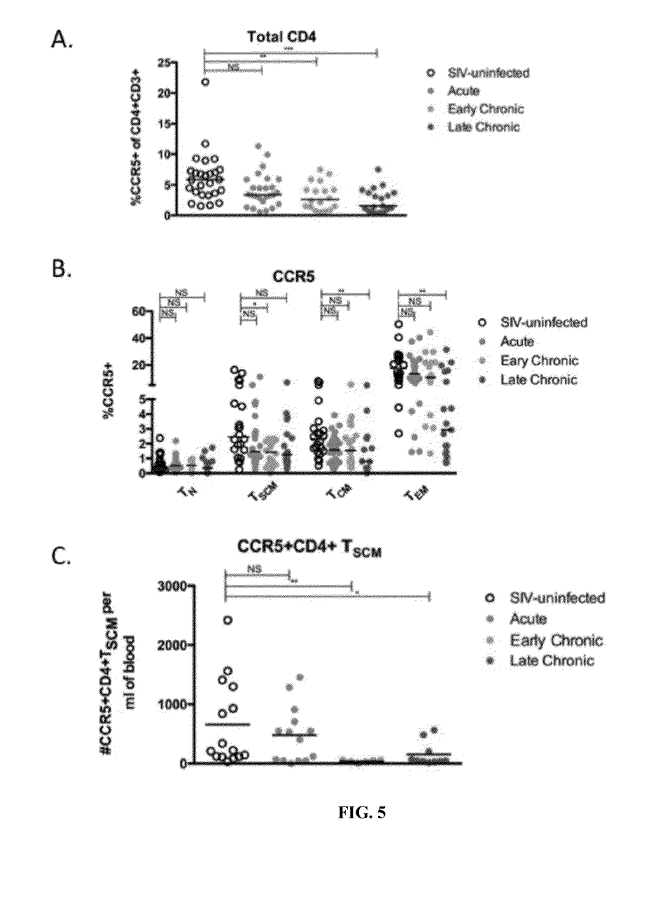

CCR5 is the main coreceptor for both HIV and SIV, and depletion of CD4⁺CCR5⁺ T cells, particularly in mucosal tissues, is a well-known hallmark of pathogenic HIV and SIV infections. In this study, the depletion of circulating CD4⁺CCR5⁺ T cells that begins in acute infection and continues during early and late chronic SIV infection of RM was confirmed (FIG. 5A). In four groups of animals, the levels of CCR5 expression were measured on the four studied subsets of CD4⁺ T cells (T_{N}, T_{SCM}, T_{CM}, and T_{EM}). As shown in FIG. 5B, the fraction of CCR5⁺ cells is significantly decreased during late chronic SIV infection of RM in both CD4⁺ T_{CM} and T_{EM} (p<0.05 and p<0.01, respectively). Interestingly, the fraction of CCR5⁺CD4⁺ T_{SCM} was significantly decreased in SIV-infected RM examined during early chronic SIV infection compared to uninfected animals (p<0.05). Although the median level of CCR5⁺CD4⁺ T_{SCM} was also decreased in late chronic SIV infection, this difference was not statistically significant when compared to SIV-uninfected animals, likely due to a wide range of values. Importantly however, a significant depletion of the absolute number of CCR5⁺CD4⁺ T_{SCM} (FIG. 5C) was found during both early and late chronic SIV infection. Together with the data shown in FIG. 4, these results indicate that pathogenic SIV infection of RM is associated with a depletion of CCR5⁺CD4⁺ T_{SCM} that occurs in the context of an overall preservation of the CD4⁺ T_{SCM} pool.

Pathogenic SIV Infection of RM is Associated with Increased Proliferation of CD4+ TSCM that Correlates Inversely with Circulating Level of CD4+ TCM

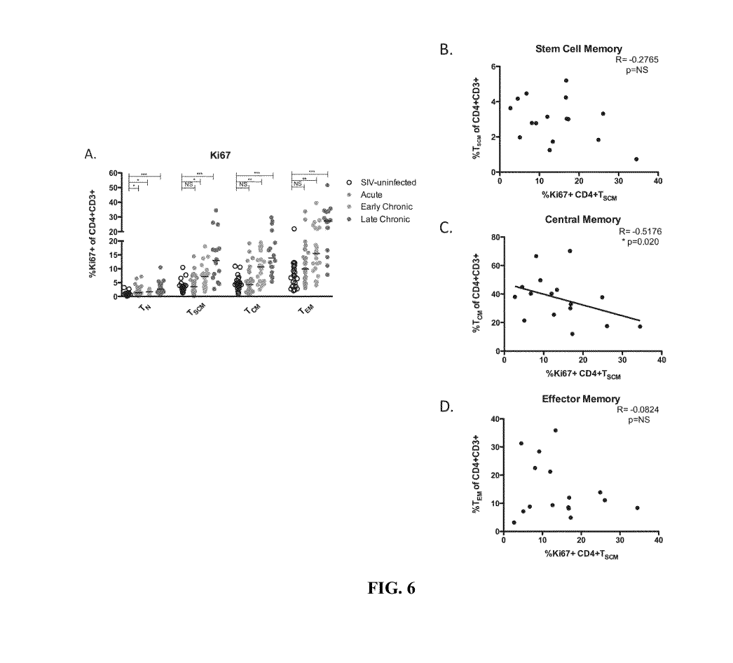

To further investigate whether and to what extent pathogenic SIV infection of RM perturbs the homeostasis of CD4⁺ T_{SCM} in our four groups of animals, the expression of the proliferation marker Ki67 in the studied subsets of CD4+ T cells (T_{N}, T_{SCM}, T_{CM}, and T_{EM}) were measured. Early and late chronic SIV infection was observed to be associated with increased proliferation of both T_{CM} and T_{EM} (FIG. 6A). Interestingly, the percentage of Ki67⁺CD4⁺ T_{SCM} was also significantly increased in SIV-infected RM examined during both early (p<0.05) and late (p<0.001) stages of infection as compared to healthy uninfected animals (FIG. 6A). The observed increase in the fraction of cycling CD4⁺ T_{SCM} could be the result of homeostatic proliferation in response to the overall depletion of memory CD4⁺ T cells, due to chronic immune activation, or both. In an attempt to assess whether homeostatic proliferation may be responsible for the increased proliferation of CD4⁺ T_{SCM} in SIV-infected RM, the relationship between the fraction of Ki67⁺CD4⁺ T_{SCM} and the levels of the four studied CD4⁺ T cell subsets was investigated. As shown in FIG. 6B-D, a significant inverse correlation between the percentage of Ki67⁺CD4⁺ T_{SCM} and the percentage of circulating CD4⁺ T_{CM} (p=0.02) was found, but not with the level of any of the other memory CD4⁺ T cell subsets. These data suggest that the increased proliferation of CD4⁺ T_{SCM} observed in SIV-infected RM represents, at least in part, a compensatory response to the CD4⁺ T_{CM} depletion induced by SIV infection.

### Dynamics of CD4⁺ T_{SCM} During Nonpathogenic SIV Infection of SM

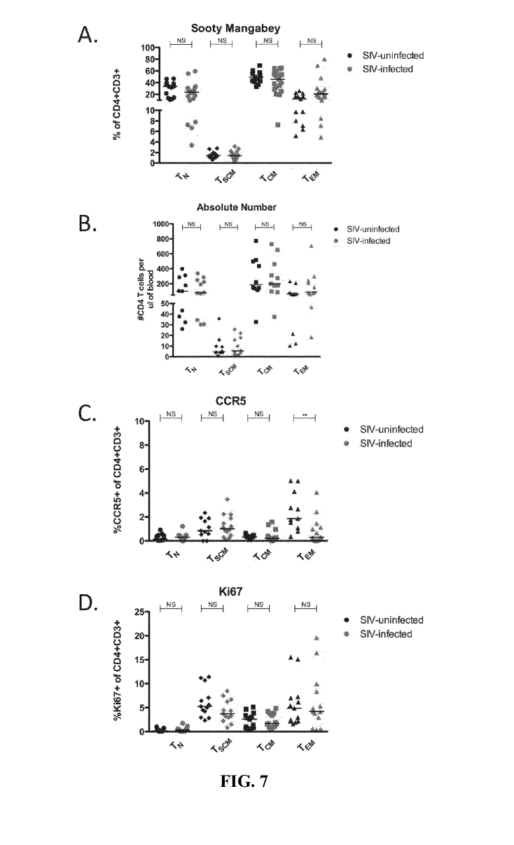

Nonpathogenic SIV infection of SM is typically associated with preserved CD4⁺ T cell counts and low levels of immune activation. How SIV infection impacts the levels of CD4+TSCM in SM Here was examined. As shown in FIG. 7A, no difference in the levels of any of the four studied subsets of CD4⁺ T cells (T_{N}, T_{SCM}, T_{CM}, and T_{EM}) was found in SIV-infected SM as compared to uninfected animals, in contrast to the depletion of T_{CM} and T_{EM} seen in chronic SIV infection of RM (FIG. 4C). Similarly, no differences were found between SIV-infected and uninfected SM with respect to the absolute number of CD4⁺ T_{SCM} per micro liter of blood (FIG. 7B). Whether SIV infection of SM is associated with a selective depletion of CD4+CCR5+TSCM was investigated, and unlike the loss of these cells in SIV-infected RM (FIG. 5B), similar levels of CD4⁺CCR5⁺ T_{SCM} were found in SIV-infected and uninfected SM (FIG. 7C). To determine whether SIV infection of SM is associated with increased proliferation of CD4⁺ T_{SCM}, the fraction of these cells expressing Ki67 were measured. As shown in FIG. 7D, no difference in the fraction of CD4⁺ T_{SCM} expressing Ki67 was found among SIV-infected and uninfected SM. Taken together, these data indicate the that nonpathogenic SIV infection of SM is characterized by overall preservation of the CD4⁺ T_{SCM} compartment, involving both CD4⁺CCR5⁻ and CD4⁺CCR5⁺ cells, and does not result in increased turnover of this memory cell subset.

### Robust Levels of CD4+TSCM Infection In Vivo are Observed in SIV-Infected RM but not in SIV-Infected SM

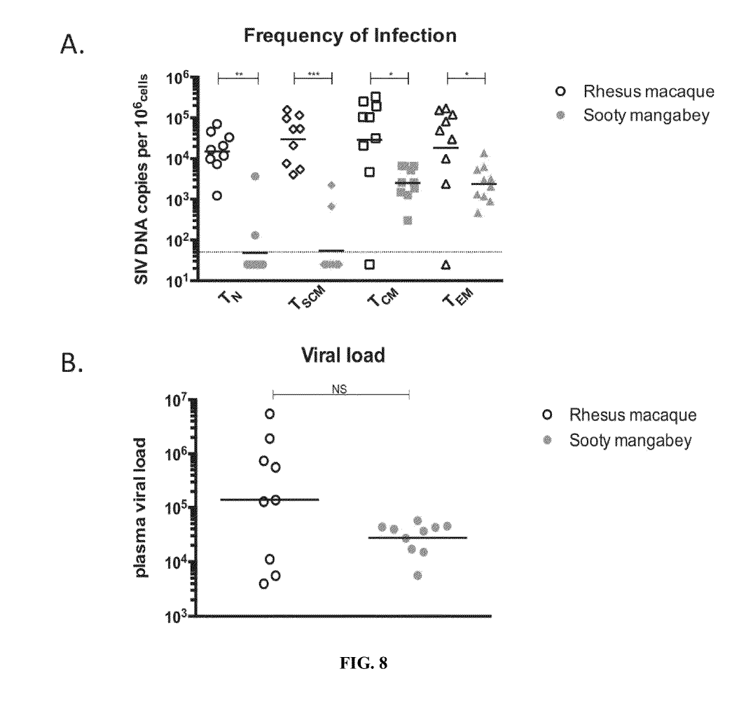

It is hypothesized that preservation of the CD4⁺ T_{CM} compartment is a key determinant of the nonpathogenic nature of SIV infection of SM. To expand upon these observations, here the levels of SIV DNA were measured in flow-cytometrically sorted samples of the four studied subsets of CD4⁺ T cells (T_{N}, T_{SCM}, T_{CM}, and T_{EM}) in nine SIVmac251-infected RM and ten SIVsmm-infected SM. For sorting T_{CM} were phenotypically defined as CD45RA⁻CD28⁺CD95⁺CCR7⁺ and T_{EM} as CD45RA⁻CD95⁺CCR7⁻. The reason for this choice was to incorporate the definition of T_{SCM} in nonhuman primates. As shown in FIG. 8A, a robust frequency of infection (i.e., greater than 1/1000 cells) was observed in CD4⁺ T_{SCM} isolated from nine out of nine SIV-infected RM. In contrast, SIV DNA levels in CD4⁺ T_{SCM} were undetectable in eight out of ten SW-infected SM. The level of SIV DNA was higher in CD4⁺ T_{N} and CD4⁺ T_{CM} of RM as compared to SM. Shown in FIG. 8B, plasma viral loads of SIV-infected RM tended to be higher than those of SM, although this trend was not statistically significant. Overall, these results indicate that in vivo infection of CD4+TSCM is frequent during pathogenic SIV infection of RM, but is absent or rare during nonpathogenic SIV infection of SM.

### Divergent CD4+ T Memory Stem Cell Dynamics in Pathogenic and Nonpathogenic SIV Infections

In this study, CD4⁺ T_{SCM} in both healthy and SIV-infected RM and SM were examined. The absolute number of CD4⁺ T_{SCM} is preserved during both pathogenic and nonpathogenic SIV infections, but SIV-infected RM showed a selective depletion of CD4⁺CCR5⁺ T_{SCM}. In SIV-infected RM, but not in SIV-infected SM, CD4⁺ T_{SCM} display significantly higher levels of proliferation that correlate inversely with both percentage and absolute number of CD4⁺ T_{CM}. Importantly, substantial levels of direct virus infection of CD4⁺ T_{SCM} were seen only in SIV-infected RM, with the majority of SIV-infected SM lacking SIV DNA within CD4⁺ T_{SCM}. Increased proliferation and infection rates of CD4⁺ T_{SCM} may play a role in the pathogenesis of SIV infection in RM. Based on their longevity and high levels of direct virus infection in pathogenic SIV-infection, CD4+TSCM may be an important site for the HIV/SIV reservoir as well as for maintaining memory T cell homeostasis.

The main results are that: (i) CD4⁺ T_{SCM} are numerically preserved during both pathogenic and nonpathogenic SIV infections, with SIV-infected RM showing a selective depletion of CD4⁺CCR5⁺ T_{SCM}; (ii) CD4⁺ T_{SCM} show significantly higher levels of proliferation that correlate inversely with the percentage of CD4⁺ T_{CM} in SIV-infected RM, but not SM; (iii) robust levels of direct virus infection of CD4⁺ T_{SCM} are found only in SIV-infected RM, with the majority of SIV-infected SM showing no evidence of CD4⁺ T_{SCM} infection. The observation that CD4⁺ T_{SCM} of healthy, SIV-uninfected RM express higher levels of CCR5 as compared to CD4⁺ T_{SCM} of healthy SM is consistent with findings in T_{CM} and a potential inherent resistance to direct infection at the virus entry level in SM.

Taken together, these data allow us to delineate a model for the role of CD4⁺ T_{SCM} in SIV pathogenesis. In SIV-infected RM, significant perturbation of the homeostasis of CD4⁺ T_{SCM} were observed in three ways, as these cells can be directly infected by the virus, are depleted in the percentage of cells expressing CCR5, and manifest increased proliferation. In contrast, none of these perturbations in the TSCM pool are present in SIV-infected SM. The significant inverse correlation between CD4⁺ T_{SCM} proliferation and CD4⁺ T_{CM} depletion observed in SIV-infected RM suggests that CD4⁺ T_{SCM} proliferate at least in part to compensate for the progressive loss of CD4⁺ T_{CM}. While the overall numeric homeostasis of CD4⁺ T_{SCM} is not altered in SIV-infected RM, it is possible that this cellular compartment loses, in time, the ability to effectively support the maintenance of CD4⁺ T_{CM}.

A striking difference between SIV-infected RM and SIV-infected SM is the level of virus infection of these cells as measured by fraction of SIV DNA positive cells. While all SIV-infected RM showed a calculated percentage of CD4⁺ T_{SCM} infection between 0.3 and 10%, eight out of ten SIV-infected SM show undetectable levels of CD4⁺ T_{SCM} infection (i.e., less than 0.005%). Given the lower levels of CCR5 expression on CD4⁺ T_{SCM} of SM as compared to RM, one possibility is that these cells are resistant to virus infection at the entry level, analogous to what has been observed for CD4⁺ T_{CM}. It should be noted, however, that other co-receptors, in addition to CCR5, as well as post-entry factors may be involved in determining the different levels of SIV infection in CD4⁺ T_{SCM} of RM and SM. Unfortunately, due to the relatively small number of CD4⁺ T_{SCM} that can be isolated from SM, we were not able to directly confirm in vitro that these cells are intrinsically more resistant to in vitro SIV infection then CD4⁺ T_{SCM} of RM.

The observation that CD4⁺ T_{SCM} are infected at high levels during pathogenic SIV infection of RM is consistent with the possibility that, once virus replication is suppressed by ART, a subset of these cells remain latently infected and may seed the previously described persistent reservoir in T_{CM}. Under these circumstances, the contribution of CD4⁺ T_{SCM} to the persistent reservoir may increase over time simply as a consequence of their enhanced proliferative ability. An intriguing corollary of this hypothesis is that, in SW-infected SM, the absence of virus infection in CD4⁺ T_{SCM} may result in an inability to maintain a persistent reservoir of latently infected CD4⁺ T cells when virus replication is suppressed by ART.

This study provides evidence that pathogenic SIV infection of RM, but not nonpathogenic SIV infection of SM, is associated with significant infection and homeostatic perturbation of CD4⁺ T_{SCM} indicating CD4⁺ T_{SCM} plays an important role both in the pathogenesis of disease progression as well as the persistence of infection under ART.

1. A method of treating or preventing a viral infection comprising administering an effective amount of a Wnt pathway inhibitor to a subject in need thereof.

2. The method of claim 1, wherein the Wnt pathway inhibitor is administered in combination with one or more an anti-viral agent.

3. The method of claim 1, wherein the subject is diagnosed with a chronic viral infection.

4. The method of claim 1, wherein the subject is diagnosed human immunodeficiency virus (HIV) 1 or 2.

5. The method of claim 1, wherein the Wnt pathway inhibitor is a small molecule inhibitor, pyrvinium, troglitazone, bosutinib, imatinib, sulindac, niclosamide, XAV-939, non-steroidal anti-inflammatory drug (NSAID), vitamin A, D, COX1, COX2 inhibitor, recombinant peptide, or antibody to a Wnt receptor or ligand thereto.

6. The method of claim 5 wherein the recombinant peptide is selected from a secreted frizzled-related protein (SFRP1, SFRP2, SFRP3, SFRP4, SFRP5), Wnt inhibitory factor 1 (Wif1), Cerberus, Sclerostin, Wise, and a Dickkopf family secreted protein (DKK-1, DKK-2, DKK-3, and DKK-4) optionally substituted with polyethylene glycol, saccharide, polysaccharide, carbohydrate or other water solubilizing moiety.

7. The method of claim 2, wherein the antiviral agent is abacavir, acyclovir, acyclovir, adefovir, amantadine, amprenavir, ampligen, arbidol, atazanavir, atripla, boceprevir, cidofovir, combivir, complera, darunavir, delavirdine, didanosine, docosanol, dolutegravir, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, stribild, tenofovir, tenofovir disoproxil, tenofovir disoproxil fumarate (TDF), tenofovir alafenamide fumarate (TAF), tipranavir, trifluridine, trizivir, tromantadine, truvada, valaciclovir, valganciclovir, vicriviroc, vidarabine, viramidine, zalcitabine, zanamivir, zidovudine, prodrugs, active metabolites, salts, alternative salts, and combinations thereof.

8. The method of claim 2, wherein the antiviral agents are emtricitabine, tenofovir DF, and efavirenz.

9. The method of claim 2, wherein the antiviral agents are emtricitabine, tenofovir DF and raltegravir.

10. The method of claim 2, wherein the antiviral agents are emtricitabine, tenofovir DF, ritonavir and darunavir.

11. The method of claim 2, wherein the antiviral agents are emtricitabine, tenofovir DF, ritonavir and atazanavir.

12. A pharmaceutical composition comprising a Wnt pathway inhibitor and one or more antiviral agents.

13. A kit comprising a Wnt pathway inhibitor and one or more an antiviral agents.
